(19) United States
(12) Patent Application Publication
(10) Pub. No.: US 2023/0389869 A1
(43) Pub. Date: Dec. 7, 2023

(54) MULTIMODAL PHYSIOLOGICAL SENSING SYSTEMS AND METHODS

(71) Applicant: MEDTRONIC, INC., MINNEAPOLIS, MN (US)

(72) Inventors: Sarah E. AHLBERG, MAPLE GROVE, MN (US); Randal C. SCHULHAUSER, PHOENIX, AZ (US)

(21) Appl. No.: 18/314,476

(22) Filed: May 9, 2023

Related U.S. Application Data
(60) Provisional application No. 63/347,864, filed on Jun. 1, 2022.

(51) Int. Cl. A61B 5/00; A61B 5/367; A61B 8/00; A61B 8/08

(52) U.S. Cl. CPC A61B 5/6801; A61B 5/0006; A61B 8/5284; A61B 8/4483; A61B 5/367

(57) ABSTRACT

A system includes a sheet flexible material having a contact surface adapted to be placed on an outer surface of a patient's body. A plurality of sensing apparatuses have respective sensing surfaces distributed across the contact surface of the sheet. One or more of the sensing apparatuses include a multimodal sensing apparatus. Each multimodal sensing apparatus includes a monolithic substrate carrying a transducer, circuitry and an electrophysiological sensor. The transducer is coupled to the circuitry and configured to at least sense acoustic energy from a transducer location of the sheet. The electrophysiological sensor is also coupled to the circuitry, and the sensor is configured to at least sense electrophysiological signals from a sensor location of the sheet, in which the sensor location has a known spatial position relative to the transducer location.

### CROSS-REFERENCE TO RELATED APPLICATION

This application claims the benefit of priority to U.S. Provisional Patent Application No. 63/347864, filed Jun. 1, 2022, which is incorporated herein by reference in its entirety.

### FIELD

The present technology is generally related to sensing physiological information, more particular to multimodal sensing technologies for use in monitoring physiological information.

### BACKGROUND

Systems exist for monitoring physiological information, including electrophysiological measurements, anatomical features, and the like. In one example, an arrangement of electrodes are placed on patient's thorax to measure cardiac electrophysiological signals. The measured electrophysiological information is combined with patient geometry to reconstruct electrophysiological signals on cardiac surface by solving an inverse problem. Typically, the patient geometry is derived from three-dimensional images acquired using computed tomography or other high-resolution imaging modality. The cost and availability of such high-resolution imaging modalities can limit the use of these and other similar technologies.

### SUMMARY

The techniques of this disclosure generally relate to multimodal sensing technologies for use in monitoring physiological information.

In one aspect, the present disclosure provides a system includes a sheet flexible material having a contact surface adapted to be placed on an outer surface of a patient's body. A plurality of sensing apparatuses have respective sensing surfaces distributed across the contact surface of the sheet. One or more of the sensing apparatuses include a multimodal sensing apparatus. Each multimodal sensing apparatus includes a monolithic substrate carrying a transducer, circuitry and an electrophysiological sensor. The transducer is coupled to the circuitry and configured to at least sense acoustic energy from a transducer location of the sheet. The electrophysiological sensor is also coupled to the circuitry, and the sensor configured to at least sense electrophysiological signals from a sensor location of the sheet, in which the sensor location has a known spatial position relative to the transducer location.

In another aspect, the disclosure provides a method that includes placing a sensing system on an outer surface of a patient's body, in which the sensing system includes an arrangement of electrophysiological sensors and ultrasound transducer modules. The method also includes providing ultrasound image data based on ultrasound images acquired by the ultrasound transducer modules according to the placement of the sensing system. The method also includes generating a three-dimensional image volume based on the ultrasound image data, in which the three-dimensional image volume includes patient anatomy and at least some of the electrophysiological sensors. The method also includes determining locations of the plurality of electrophysiological sensors and at least one anatomical surface within the patient's body based on image processing applied to the three-dimensional image volume. The method also includes generating geometry data representative of a spatial relationship between the electrophysiological sensors and the anatomical surface in a three-dimensional coordinate system.

In another aspect, the disclosure provides a system that includes a sensing system and a remote system. The remote system can be coupled to the sensing system through a communication link. The sensing system includes an arrangement of electrophysiological sensors and ultrasound transducer modules on a flexible sheet adapted to be placed on an outer surface of a patient's body. The electrophysiological sensors are configured to measure electrophysiological signals from the body surface, and the ultrasound transducer modules configured to measure acoustic waves from the body surface and provide respective ultrasound images. The remote system includes one or more non-transitory machine readable media to store data and instructions. The data includes ultrasound image data representative of the respective ultrasound images provided by the ultrasound transducer modules, electrophysiological data representative of the electrophysiological signals measured from the body surface, and geometry data representing a spatial relationship between the electrophysiological sensors and patient anatomy in a three-dimensional coordinate system, the geometry data being determined based on the ultrasound image data. The remote system also includes a processor to access the media and execute the instructions, such as to analyze at least one of the ultrasound image data and the electrophysiological data, and provide output data to visualize physiological information for the patient based on the analysis.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF DRAWINGS

FIG. 1 is a schematic diagram of part of a sensing system that illustrates an example multimodal physiological sensing apparatus.

FIG. 2 is a side view of the sensing apparatus of FIG. 1.

FIG. 3 is a schematic diagram of part of a sensing system that illustrates another example multimodal physiological sensing apparatus.

FIG. 4 is a side view of the sensing apparatus of FIG. 3.

FIG. 5 is a top view of an example multimodal physiological sensing apparatus implemented on a circuit board.

FIG. 6 is a bottom view of the multimodal physiological sensing apparatus of FIG. 5.

FIG. 7 is a top view of an integrated ultrasound sensor transducer module that illustrates a plurality of transducer elements.

FIG. 8 is a cross-sectional view that illustrates part of the transducer module of FIG. 7.

FIG. 9 is a cross-sectional view that illustrates an example electrode.

FIG. 10 is an example sensing system that includes an arrangement of multimodal physiological sensing apparatuses and electrodes.

FIG. 11 is another example sensing system that includes an arrangement of multimodal physiological sensing apparatuses.

FIG. 12 is a perspective view of a wearable sensing system that includes an arrangement of multimodal physiological sensing apparatuses.

FIG. 13 illustrates a sensing system wirelessly coupled with a remote monitoring system.

FIG. 14 illustrates a sensing system coupled with a remote monitoring system.

FIG. 15 is a conceptual diagram that illustrates communication between sensors that can be implemented as part of sensor calibration.

FIG. 16 is a block diagram that illustrates a method to determine geometry data.

FIG. 17 is a block diagram that illustrates an example of an analysis and treatment system.

FIG. 18 is a block diagram that illustrates an example of part of the remote system shown in FIG. 17.

FIG. 19 depicts an example of outputs that can be generated by the system of FIG. 17 or 18.

### DETAILED DESCRIPTION

This description relates to systems and methods to implement multimodal sensing for use in measuring and/or monitoring physiological information. The sensed physiological information can be combined and analyzed to a measure of one or more physiological conditions. The systems and method described herein can be used to measure the one or more physiological conditions for diagnostic purposes. Additionally, or alternatively, the systems and methods described herein can be used during or in connection with an intervention, such as to measure of one or more physiological conditions as part of (e.g., before, during and/or after) delivery of a therapy to the patient and/or performing a surgical intervention.

For example, a sensing system includes a sheet of flexible material adapted to be applied to and conform to an outer surface of patient's body. The sheet can be in the form of garment, such as vest or shirt or hat, which can be worn by the patient so that a contact surface of the sheet can engage the outer surface of the body. A distributed arrangement of sensors are carried by the sheet. Thus when the sheet is applied (or worn) to the outer surface of the body, the sensors are adapted to sense more than one type physiological information from the body. In an example, the sensors include an arrangement of electrodes and audio transducers (e.g., ultrasound and/or auscultation transducers), in which at least some of the sensors are implemented in multimodal sensing modules.

For example, a multimodal sensing apparatus includes an electrode and a transducer integrated in a monolithic structure (e.g., an integrated circuit (IC) chip, system on chip (SoC) or mounted to a circuit board substrate). The transducer can be a solid state ultrasound or auscultation transducer implemented on or within a packaging material. The transducer can include a number of transducer elements coupled to electrical circuitry also implemented within the packaging material (e.g., on chip). The electrical circuitry is configured to control the transducer elements to transmit and receive signals and to process the received signals (e.g., amplification and filtering) to provide physiological signals. The electrical sensor (e.g., an electrode) can also be implemented on or partially within the packaging material, and have a spatial position that is known relative to the transducer. The physiological information acquired by the sensors can be communicated from the respective sensing apparatuses to a remote device through a communication link (e.g., a wireless or physical link) for storage and/or additional processing, such as described herein.

Respective image volumes generated by ultrasound transducers can be stitched together to provide a compounded (three-dimensional) image volume for the body, which can be used to generate geometry data representative of internal anatomy (e.g., one or more cardiac surfaces, lungs and bones) as well as the body surface over which the sensing system is placed. The 3D compounded image volume for the body further can be stitched together over time produce a four-dimensional anatomical image (e.g., a motion picture of anatomy) in real-time for the patient. The geometry data can also be combined with electrophysiological data representing electrophysiological signals sensed by electrodes distributed across the patient's body to generate one or more electro-anatomical maps. The electro-anatomical maps can display a visualization of real-time electrophysiological information (e.g., body surface and/or reconstructed electrophysiological signals) superimposed on the four-dimensional anatomical image of the heart, which can also be a live real time image of the heart. Advantageously, the patient workflow can be performed in the absence of ionizing radiation (e.g., without any CT, fluoroscopy or other x-ray imaging). Because the approach can be “fluoro-free,” patient as well as the caregivers need not be exposed to such radiation. Additionally, because both anatomical data and electrophysiological data can be obtained using a single sensing apparatus (e.g., in the form of a sheet or garment, such as a vest), the time and cost associated with collecting such information as well as generating anatomic and/or electrophysiological graphical maps can be reduced compared to many existing approaches.

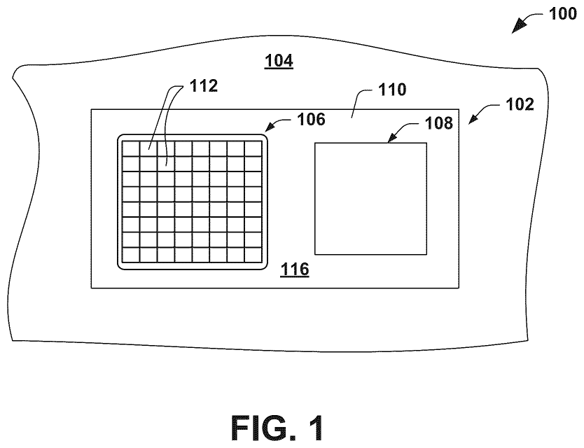

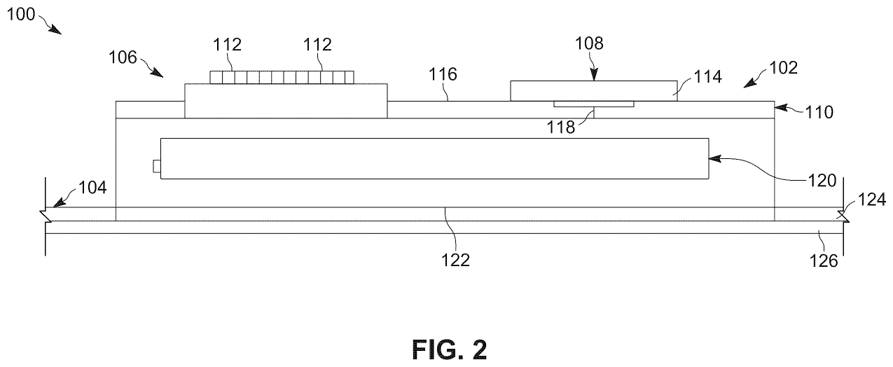

FIGS. 1 and 2 depict part of a sensing system 100 that includes an example multimodal physiological sensing apparatus 102. FIG. 1 shows a top view of the sensing apparatus 102 on a sheet of flexible material 104, and FIG. 2 shows a side cross-sectional view of the sensing apparatus. While a single sensing apparatus 102 is shown attached to a portion of the sheet 104 in FIG. 1, the sheet will typically be configured to cover and conform to a region of interest on a patient's body, and a plurality of instances of the sensing apparatus 102 will be attached to the sheet 104 in distributed arrangement with respective sensing surfaces exposed at the contact surface of the sheet. As described herein, the sheet 104 can be a stretchable, conformable material in the form of a wearable garment, such as a vest, shirt or wrap, or a patch configured for placement on a region of interest. Additional sensors and electronics can also be attached to or implemented in the sheet 104 according to application requirements. The sheet 104 can hold each instance of sensing apparatus 102 in contact with an outer surface of the body surface when placed on the patient's body. Alternatively or additionally, the sheet 104 can be held in place by one or more straps and/or an adhesive material can be used to maintain contact between the sensing apparatus 102 and the outer surface of the body. The sheet 104 thus can provide single apparatus configured to collect more than one type of physiological data, such as including image data, acoustic data and electrophysiological data. As a result, collecting such multi-modal physiological data can be facilitated compared to many existing approaches.

The sensing apparatus 102 includes a transducer module (e.g., a solid state transducer module) 106 and an electrophysiological sensor 108. As shown in the example of FIGS. 1 and 2, the sensing apparatus 102 is a monolithic structure in which the transducer module 106 and sensor 108 are carried by a common substrate material 110. For example, the sensing apparatus 102 is implemented as an integrated circuit (IC) chip. Thus, the transducer module 106 and sensor 108 can be fabricated as an integrated structure using IC fabrication methods, in which the substrate is formed of a packaging material (e.g., epoxy molding compound, magnetic molding compound, polyimide, metal, plastic, glass, ceramic, etc.). The packaging material can encapsulate an IC die, which includes the transducer 106, the sensor 108 and/or associated circuitry 120 therein.

As an example, the sensor 108 includes an electrode 114 having one or more layers of an electrically conductive material (e.g., aluminum, silver, gold, copper etc.) that extend from a surface 116 of the substrate 110. For example, the electrode 114 can be formed by a deposition process through a patterned mask structure (e.g., by evaporation or sputtering). Metal interconnects, shown schematically at 118, also can be formed in the substrate 110 to couple the electrode 114 to associated circuitry formed in the substrate 110. The circuitry 120, shown schematically as 120, can be configured to amplify and/or filter electrophysiological signals sensed by the electrode 114.

In some examples, the circuitry 120 includes wireless interface configured to send and receive signals relative to sensing apparatus 102, such as through a wireless communication link between the sensing apparatus and a remote unit. The signals sent from the sensing apparatus 102 can include electrophysiological signals sensed by the electrode 114 and/or signals measured by the transducer module 106. The signals can be raw signals or processed signals, such as have been processed by control and signal processing electronics implemented as part of the circuitry 120.

In other examples, interconnects within the substrate 110 can also, or alternatively, couple the electrode 114 and the circuitry 120 (e.g., through respective interconnects) to an arrangement of output terminals 122 (e.g., contacts or pins) formed at a mounting surface of the substrate 110. The terminals 122 thus are adapted for sending and/or receiving signals (and/or electrical power) relative to the sensing apparatus 102. The configuration and arrangement of terminals 122 can vary according to the type of IC packaging used to form the sensing apparatus 102. The terminals 122 can couple to respective pads or contacts (not shown) implemented in the sheet 104. For example, the sheet 104 can include multiple layers 124 and 126. The pads or contacts can be formed on a surface of layer 126, which includes traces and/or wires configured to carry the electrical signals and/or power relative to the sensing apparatus 102. The other layer 124 can provide flexible cover over the entire layer 126 or over a portion that includes the traces and/or wires. The traces and/or wires can route to respective connectors provided at one or more locations of the sheet 104. The connectors can be coupled to a remote unit for further processing and/or analysis of the signals. The remote unit can also provide control instructions to the sensing apparatus 102 through the communication link, which includes the wires and/or traces.

In an example, the transducer module 106 can be implemented as an ultrasound transducer and/or an auscultation transducer. For example, the transducer module 106 is implemented as a transducer array having a number of transducer elements 112 distributed across the surface of the apparatus 102. Each of the transducer elements 112 can be formed as a microelectromechanical systems (MEMs) acoustic transducer element (e.g., a capacitive micromachined ultrasound transducer) configured to receive and/or transmit acoustic energy, such as acoustic waves that propagate through the body. As used herein, the acoustic waves can include audible sound waves and/or ultrasound waves propagating through the body.

For example, each of the MEMs elements is configured to transmit ultrasonic waves as well as to receive ultrasonic vibrations (e.g., about 10 KHz to about 100 MHz) greater which are converted to electronic signals, amplified, and processed by associated circuitry 120. The circuitry 120 can also convert the signals from the transducer elements 112 into electrical signals representative of a corresponding ultrasound image volume, which can vary over time (e.g., a 3D or 4D image volume).

In another example, the transducer is implemented as or includes an auscultation device configured to receive audible acoustic vibrations (e.g., about 10 Hz to about 20 KHz) which are converted to electronic signals, amplified, and processed by associated circuitry 120. The electronic signals can be communicated to a remote unit through a communication link (e.g., wireless or through a physical medium), such as described herein.

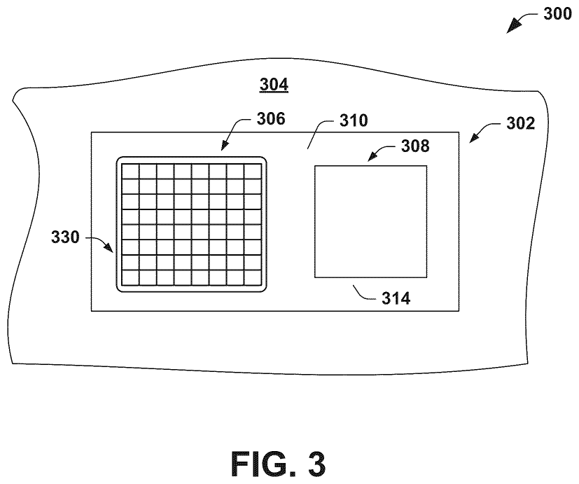

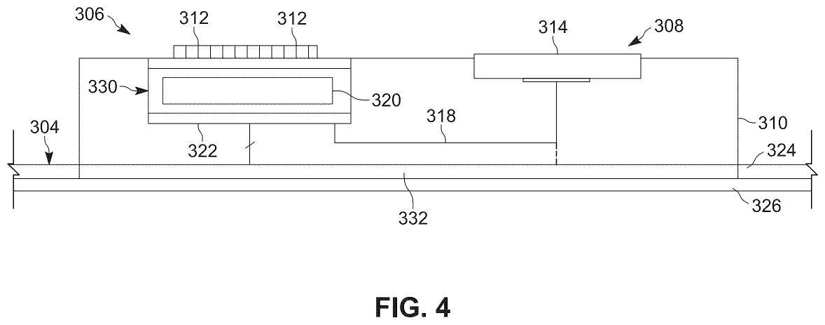

FIGS. 3 and 4 depict part of a sensing system 300 that includes an example multimodal physiological sensing apparatus 302. FIG. 3 shows a top view of the sensing apparatus 302 on a sheet of flexible (e.g., stretchable and conformable) material 304, such as described herein. FIG. 4 shows a side cross-sectional view of the sensing apparatus 302. As mentioned above, a plurality of instances of the sensing apparatus 302 typically will be attached to the sheet 304 in distributed arrangement with respective sensing surfaces exposed at a contact surface of the sheet for acquiring physiological information from a patient's body where the sheet and sensing apparatuses 302 are positioned.

Similar to the example of FIGS. 1 and 2, the sensing apparatus 302 includes a transducer module 306 and an electrophysiological sensor 308, shown as an electrode structure 314. In the example of FIGS. 3 and 4, however, the sensing apparatus 302 is implemented as system on chip (SoC), in which at least some of the transducer 306, sensor 308 and associated circuitry are formed as separate (discrete) components that are assembled together in common monolithic substrate 310 (e.g., packaging material or other suitable electrically insulating material) to provide a monolithic SoC sensing apparatus 302. As an example, the transducer module 306 and associated circuitry (e.g., control and signal processing electronics) 320 can be formed on a respective transducer IC die 330, and the sensor 308 and other components (e.g., discrete circuit components, IC die, etc.) can be coupled to one or more bond pads of the IC die using respective bond wires. In the example of FIG. 4, a bond wire 318 couples the electrode 314 to a bond pad(s) 322 of the transducer die 330. Once assembled and connected together, the packaging material 310 can be applied to encapsulate portions of the circuit and hold the SoC structure together.

In one example, the SoC apparatus 302 includes a wireless interface configured to send and receive signals relative to sensing apparatus 302, such as through a wireless communication link between the sensing apparatus and a remote unit. The wireless communication link can be a bidirectional link or a unidirectional link. The wireless interface can be implemented as part of the circuitry 320 on the transducer IC die or another IC die within the SoC apparatus 302. The signals communicated through the communication link can include electrophysiological signals sensed by the electrode 314 and/or signals obtained by the respective elements of the transducer module 306. The signals can include raw signals and/or processed signals, such as have been processed by control and signal processing electronics implemented as part of the circuitry 320. Control instructions can also be received by the wireless interface through the wireless communication link.

In another example, the SoC apparatus 302 can further include an arrangement of terminals 332 (e.g., contacts or pins or pads) formed at a mounting surface of the substrate 310. The transducer IC bond pads 322 are coupled to respective terminals 332 through bond wiring or other connections. The terminals 332 can be input/output terminals adapted for sending and/or receiving signals (and/or electrical power) relative to the sensing apparatus 302. The configuration and arrangement of terminals 332 can vary according to the type of IC packaging used to form the sensing apparatus 302. The terminals 332 can also couple to respective pads or contacts (not shown) implemented in the sheet 304. For example, the sheet 304 can include multiple layers 324 and 326. The pads or contacts can be formed on a surface of layer 326, which includes traces and/or wires configured to carry the electrical signals and/or power relative to the sensing apparatus 302. The other layer 324 can provide flexible cover over the entire layer 326 or over a respective portion that includes the traces and/or wires. The traces and/or wires can route to respective connectors provided at one or more locations of the sheet 304. The connectors can be coupled to mating connector adapted to be coupled to a remote unit (not shown) for further processing and/or analysis of the signals. The remote unit can also provide control instructions to the sensing apparatus 302 through the communication link, which includes the wires and/or traces.

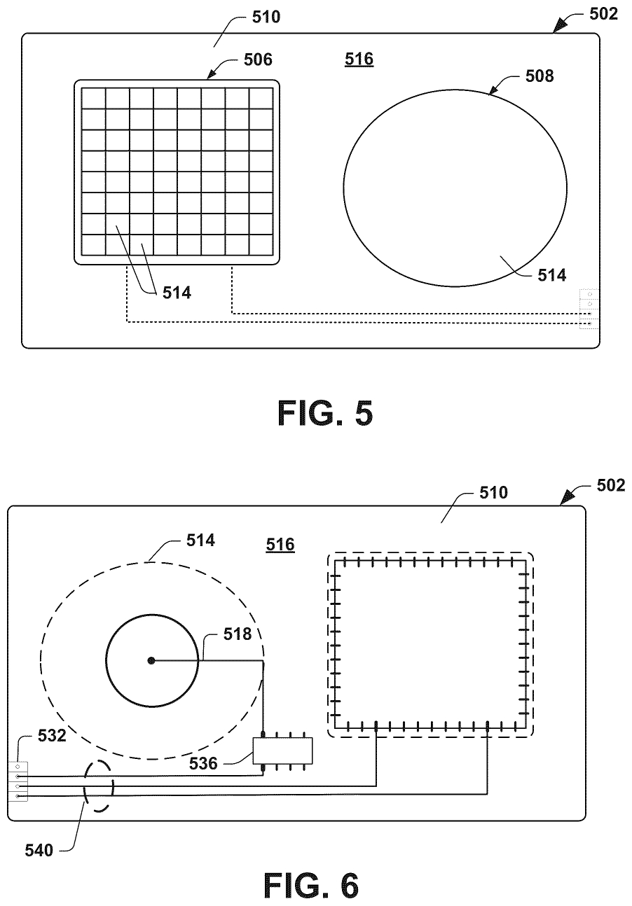

FIGS. 5 and 6 illustrate another example multimodal physiological sensing apparatus 502 implemented on a printed circuit board (PCB) substrate 510, such as flexible insulating substrate (e.g., formed of a polyimide (PI) film, a Polyester (PET) film, or polytetrafluoroethylene (PTFE) film. FIG. 5 shows a top view and FIG. 6 shows a bottom view of the sensing apparatus 502 implemented on the flexible PCB substrate 510. As described herein, a plurality of instances of the sensing apparatus 502 can be mounted (e.g., by adhesive, clamps, fittings or the like) to a sheet of stretchable and conformable material to provide a sensing system.

The sensing apparatus 502 can be configured similar to the example of FIGS. 1-4. For example, the sensing apparatus includes a transducer module 506 and an electrophysiological sensor 508. The transducer module 506 can be implemented as a transducer IC chip that is coupled to the top surface 516 of the PCB substrate 510 by pins or bond pads soldered to pads of the PCB substrate. The PCT substrate 510 can include a number of layers, which can include traces or other circuitry configured for routing signals and power used during operation of the sensing apparatus 502. As used herein, the substrate 510 is a monolithic substrate configured to carry the transducer module 506, the sensor 508 and circuitry, which can be integrated with the transducer (e.g., in an IC) and/or be separately mounted to the substrate.

For example, the transducer module 506 is implemented as a transducer IC chip that includes an array of transducer elements 512 coupled to circuitry also implemented within the transducer IC chip. The transducer elements 512 can be MEMs ultrasound transducer elements, piezoelectric ultrasound elements or auscultation transducers. The transducer IC chip can include electrical circuitry configured to control the transducer elements to transmit and receive ultrasound signals and to process the received signals (e.g., amplification and filtering) to provide respective physiological signals.

The electrical electrophysiological sensor 508 can be implemented as an electrode 514 that is also mounted to the surface 516 of the PCB substrate 510. In an example, the electrode 514 includes a coupling (e.g., pad or terminal) coupled to circuitry 536 by electrical trace or wires 518. The circuitry 536 can be configured to process (e.g., amplify and filter) the electrophysiological signals acquired by the electrode 514. In another example, the electrode 514 is coupled to a terminal (or terminals) of an IC carrying the transducer module 506, which includes circuitry to process the electrophysiological signals acquired by the electrode 514. The circuitry can also include a communication interface to communicate signals relative to the sensing apparatus 502.

In example where the sheet to which the sensing apparatuses includes connectors and/or circuitry for further processing or communication of the acquired signals, the communication interface can be coupled to respective terminals of a connector 532, such as by traces and/or wires 540 route through one or more layers of the PCB substrate 510. The connector 532 can include input/output terminals adapted for sending and/or receiving signals (and/or electrical power) relative to the sensing apparatus 502 (e.g., through the communication interface). The connector 532 can be adapted to couple to respective pads or contacts (e.g., of a mating connector) implemented at respective locations of the sheet (not shown), which mating connectors are adapted to be coupled to a remote unit for further processing and/or analysis of the signals. The remote unit can also provide control instructions to the sensing apparatus 302 through a respective communication link, which includes the wires and/or traces. One or more sheet couplings can also be configured to hold the sensing apparatus at a fixed location with respect to the sheet.

In another example, the sensing apparatus 502 includes a wireless communication interface coupled to the PCB substrate 510, such as implemented in circuitry 536 or the transducer IC. The wireless interface can be configured to send and receive signals relative to sensing apparatus 502, such as through a wireless communication link between the sensing apparatus and a remote unit. The signals sent from the sensing apparatus 102 can include electrophysiological signals sensed by the sensor 508 and/or signals (e.g., acoustic energy) measured by respective elements of the transducer module 506. The signals can be raw signals or processed signals, such as have been processed by control and signal processing electronics, such as implemented as part of the circuitry 120. In an example that uses a wireless communication interface, the sensing apparatus can use an internal power supply such as a battery and/or electrical power can be supplied through respective power terminals (e.g., implemented through the connector 532).

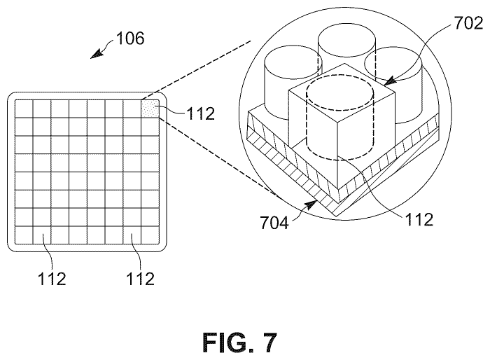

FIG. 7 depicts an example of an integrated ultrasound sensor transducer module 106 that includes a plurality of MEMs transducer elements 112 of FIGS. 1 and 2. Accordingly, the description of FIG. 7 also refers to FIGS. 1 and 2. An enlarged diagrammatic view of an example MEMs element 112 is also shown in FIG. 7. The MEMs transducer element 112, for example, includes a speaker and microphone structure, shown at 702, formed on a semiconductor structure 704 and configured to convert received acoustic energy (e.g., ultrasonic vibrations at frequencies of about 10 KHz to about 100 MHz) to electronic signals. The semiconductor structure 704 can include integrated circuitry formed therein, which is configured to process the electronic signals from the speaker and microphone structure 702 (e.g., amplification, filtering and image conversion) and convert such signals into corresponding ultrasound image volumes, which can vary over time (e.g., a 3D or 4D image volume). In another example, the speaker and microphone structure 702 is implemented as an auscultation device configured to receive audible acoustic vibrations (e.g., about 10 Hz to about 20 KHz) and convert the vibrations to electronic signals, which can be amplified and processed by associated circuitry of the semiconductor structure 704.

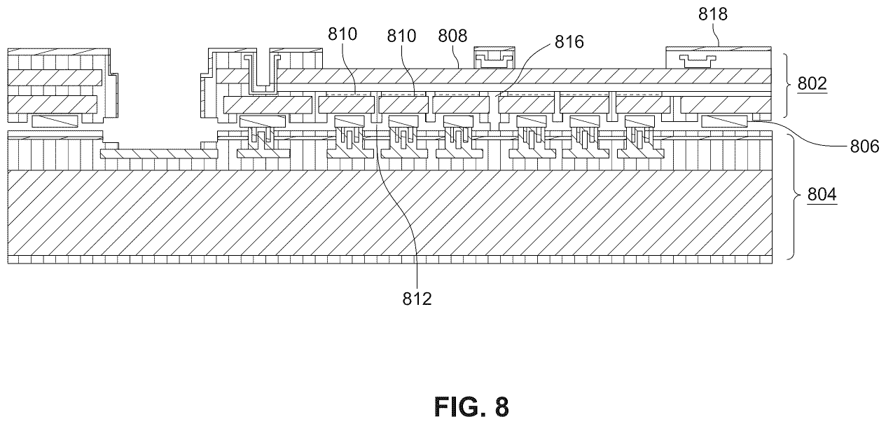

FIG. 8 is a cross-sectional view that illustrates part of the MEMs transducer module 106 of FIG. 7, such as can be fabricated by bonding a MEMs wafer module 802 with a CMOS wafer module 804 (e.g., using a metal bonding method). In an example, the MEMs module 802, such as including an array of the MEMs microphone structures 702, is formed from bulk silicon (Si) and silicon-on-insulator (SOI) wafers to provide respective sealed cavities of the MEMs module. The module 802 can include metal bond pads 806 along a periphery of the module to couple the MEMs transducers to associated circuitry implemented in the CMOS wafer module 804. The MEMs wafer module 802 can include a MEMs active transducer membrane 808 is formed over MEMs support membrane 810. The MEMs support membrane is formed over an electrical isolation region 812. A cavity 814 is formed between the active transducer membrane 808 and the support membrane 810 into and out of which the active membrane can deflect. An isolation trench 816 can be formed between adjacent transducer elements 112. The chip module 106 can also include an arrangement of interconnects (e.g., formed of metallization layer) for coupling to the respective transducer elements. Each element can be individually electrically connected and separately addressable (and controllable) from the circuitry implemented in the CMOS wafer module 804.

An example of MEMs ultrasound transducers that can be used to implement the transducer module 106 and associated circuitry is disclosed in Ultrasound-on-Chip platform for medical imaging, analysis, and collective intelligence Proc Natl Acad Sci USA, 2021 Jul. 6; 118 (27), which is incorporated herein by reference. Other types and configurations of ultrasound transducers can be used in other examples.

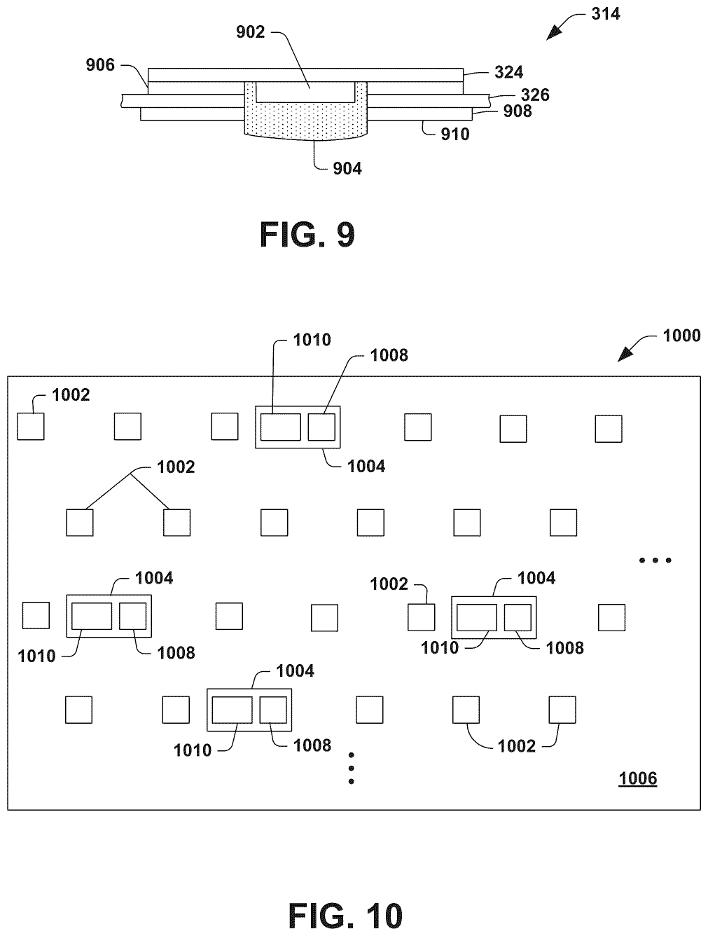

FIG. 9 is a cross-sectional view that illustrates an example electrode, such corresponding to electrode 314 of FIGS. 3 and 4. The electrode structure 314 includes a first electrode layer 902 of one or more electrically conductive materials, which can be formed (e.g., deposited) onto a contact surface of a substrate layer 324. As an example, the electrode layer can be formed of a silver or silver alloy material (or other electrically conductive materials, such as copper, copper alloys or silver alloys to name a few).

A layer 904 of an electrically conductive gel (or other pliant and electrically conductive material) can be deposited over the electrode layer 902. For example, the layer 904 can be an adhesive gel (e.g., wet gel or a solid gel construction), which can applied by the manufacturer (e.g., before shipping) or the layer 904 can be applied prior to use (e.g., by the user). In another example, the layer 904 could be dry electrode structure. The layer 904 and the electrode 902, individually or collectively, form the electrode structure 314 that provides an electrically conductive interface configured to contact with a body surface of the patient.

An insulating layer 906 can be provided on a contact surface of the substrate layer 324 to cover the electrically conductive traces applied with the layer 902. The insulating layer can be a dielectric material having a high dielectric constant sufficient to prevent the flow of electrical current. The insulating layer 906 can be a coating that can be applied as a liquid or (e.g., via spraying, deposition, or the like) onto the contact surface of the flexible substrate layer 324 and over the exposed electrically conductive traces. The insulating layer 906 can be applied to the entire contact surface except where the electrode layers 902 and 904 have been applied to the substrate layer 324. A mask or other means can be utilized to prevent application of the insulating material onto the exposed electrode structures 902.

A corresponding adhesive layer 908 can be applied in a circumscribing relationship around each the electrode layers 902 and 904 to facilitate secure attachment of the electrode structure 314 to a patient's body surface. For example, the adhesive layer 908 can be in the form of an annular ring of a foam or fabric material that surrounds each the electrode structure 314. For example, the layer 906 can be secured to the elastic conformable layer 326 via an appropriate adhesive layer. The adhesive layer can be formed as an integral part of the layer 906 itself or be applied separately. Alternatively, the annular ring can formed from a sheet of a material having one side surface 910 containing a medical grade adhesive while the other side can be initially free of adhesive, but can be affixed to the contact surface side of the elastic polymer layer by applying an adhesive layer. The adhesive can be the same adhesive that is used to affix the polyester layer to the stretchable fabric layer 326 or it can be different.

Other example electrode configurations could be used to provide the electrophysiological sensor. For example, the electrophysiological sensors can be implemented as dry foam electrodes or dry polymer-based electrodes. An example of a dry polymer-based electrode structure that can be used is disclosed in I. Wang et al., “A Wearable Mobile Electrocardiogram measurement device with novel dry polymer-based electrodes,” TENCON 2010-2010 IEEE Region 10 Conference, 2010, pp. 379-384, doi: which is incorporated herein by reference.

FIGS. 10, 11 and 12 depict examples of multimodal sensing systems 1000, 1100 and 1200 having different configurations, which can be selected and used according to application requirements. Each of the sensing systems 1000, 1100 and 1200 includes an arrangement of sensing apparatuses, which can be implemented according to any one or more of the example configurations shown and/or described herein (e.g., FIGS. 1-9).

FIG. 10 depicts an example multimodal sensing system 1000 in the form of a generally planar sheet having an arrangement of sensing apparatuses 1002 and 1004 distributed across a sheet (e.g., a patch or a panel) of flexible, conformable material, shown as 1006. The size and shape of the sheet 1006 can depend on the region of interest of a patient's body where the sheet is adapted to be placed. The number and spatial density of sensing apparatuses 1002 and 1004 can also be configured according to sensing requirements. As an example of FIG. 10, the sensors 1002 are configured as electrodes adapted to sense electrophysiological signals from a patient's body. The sensing apparatuses 1004 are configured as integrated multimodal sensing apparatuses (e.g., apparatuses 102, 302 or 502) including an electrode 1008 and transducer 1010. The electrode 1008 is configured to sense electrophysiological signals from a patient's body. The transducer 1010 can be configured as an ultrasound and/or auscultation transducers, such as described herein. In the example of FIG. 10, there are a greater number of the electrodes 1002 than the number of integrated multimodal sensing modules 1004. As an example, there can be two times or more electrodes 1002 than the number of integrated multimodal sensing modules 1004. As another example, there can be one of the integrated multimodal sensing modules 1004 for every 4, 8 or 16 electrodes 1002 on the sheet 1006. Other relative numbers and spatial densities of sensing apparatuses 1002 and 1004 can be used in other examples.

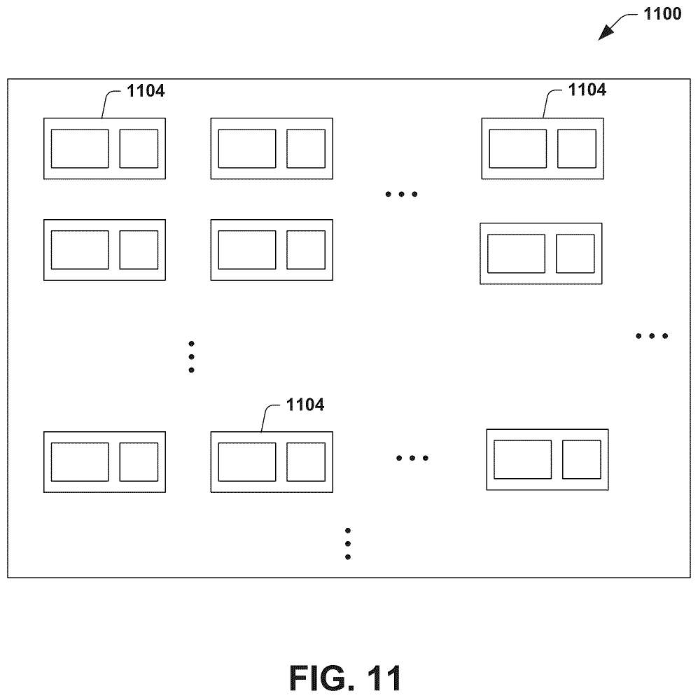

For example, FIG. 11 depicts a configuration of multimodal sensing system 1100 in which only as integrated multimodal sensing apparatuses 1104 are distributed across a sheet (e.g., a patch or a panel) of flexible, conformable material, shown as 1106. Each of the sensing apparatuses 1104 can be implemented as a respective instance of the multimodal sensing apparatus 102, 302 or 502. As described with respect to FIG. 10, the size and shape of the sheet 1006 can depend on the region of interest of a patient's body where the sheet is adapted to be placed. The number and spatial density of sensing apparatuses 1104 can also be configured according to sensing requirements. In an example, each of the sensing apparatuses 1104 includes an electrode 1008 and ultrasound and/or auscultation transducer 1010, such as described herein.

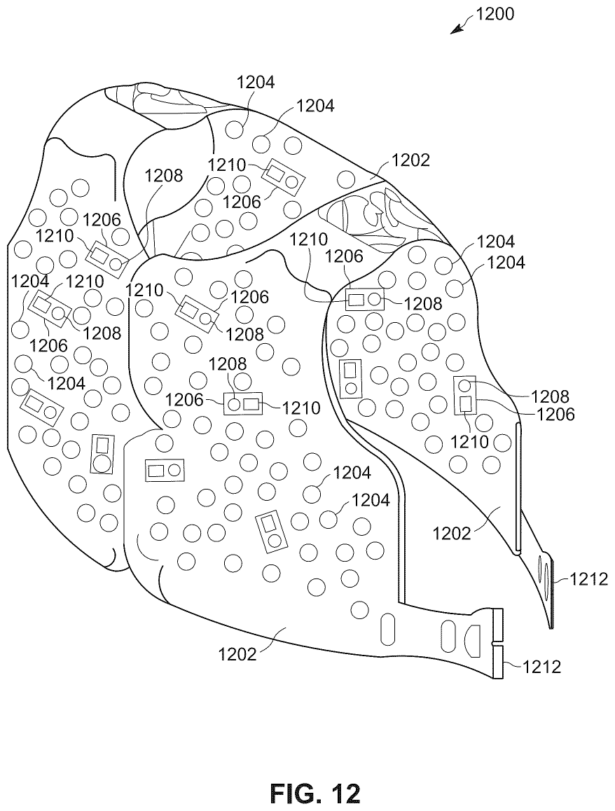

FIG. 12 depicts an example of sensing system 1200 in the form of a garment (e.g., vest, shirt, hat or other wearable clothing) adapted to be applied to a torso of a patient (e.g., a human patient); however, different configurations can be utilized depending on the patient (e.g., could be human or other animal) and the particular types of physiological information to be acquired. The system 1200 can come in a plurality of sizes to accommodate a range of patient's sizes and body types.

The sensing system 1200 includes one or more sheets of flexible, conformable material 1202 that provides a sensor-carrying substrate. For example, a single sheet can be formed in the desired shape such as shown in FIG. 12 or multiple sheets can be connected together to provide the desired shape. The flexible sheet 1202 can include one or more layers of a stretchable material, such as a woven or non-woven fabric material that exhibits high elasticity, such as spandex or elastane, although other elastic panels of conformable material can be utilized (e.g., similar to that used in some athletic clothing). The stretchable fabric layer can be formed of a synthetic, natural or combination of synthetic and natural materials. The sheet 1202 is configured to allow sections and the entire substrate to be highly conformable to the patient's body shape and movements. The conformable sheet 1202 can exhibit an amount of stretch to maintain a maximum distance between adjacent electrodes within a predetermined distance horizontally (e.g., about 5 to 10 cm) and vertically (e.g., about 3 to 7 cm) when applied to a patient's body. The sheet 1202 can be formed from a substantially planar sheet of flexible material that can bend and/or twist in directions transverse from its planar configuration. The flexible sheet 1202 also provides sufficient structure to maintain general spatial distribution of the sensing apparatuses 1204 and 1206 distributed across the sheet.

In the example of FIG. 12, the sensing apparatuses 1204 are configured as electrodes adapted to sense electrophysiological signals from an outer surface of patient's body. The other sensing apparatuses 1206 are configured as integrated multimodal sensors (e.g., instances of sensing apparatus 102, 302 or 502), each including an electrode 1208 and a transducer 1210. The electrode 1208 is configured to sense electrophysiological signals from a patient's body. The transducer 1210 is configured as an ultrasound and/or auscultation transducer, such as described herein. The sensing apparatuses 1206 can be implemented as an IC or SoC with circuitry configured to control and process the physiological signal measured from the outer surface of the body where the sensor is positioned.

In some examples each of the sensing apparatuses 1204 and 1206 can be coupled to a layer of the sheet configured to carry wires, traces and/or electrical circuitry (not shown in FIG. 12). For example, electrical traces and/or wires electrically couple each of the sensing apparatuses 1204 and 1206 to respective terminals of a connector 1212. There can be a number of one or more connectors 1212 configured to couple with mating connectors to carry signals to/from a remote unit. The traces/wires can carry electrical signal and/or power relative to and/or from respective sensing apparatuses 1204 and 1206 and remote unit, depending on the configuration of the sensors. In another example, the sensors 1206 include wireless interfaces configured to wirelessly communication signals to and from the respective sensing apparatuses 1204 and 1206. The signals communicated from the sensing system 1200 (e.g., wirelessly or through a physical media) can include one or more physiological signals acquired from the body surface, such as electrophysiological (e.g., electromyography (EMG), electrocardiography (EKG), electroencephalography (EEG)) signals or auscultation signals over time. The signals can also include ultrasound signals representative of ultrasound images acquired by ultrasound transducers over time.

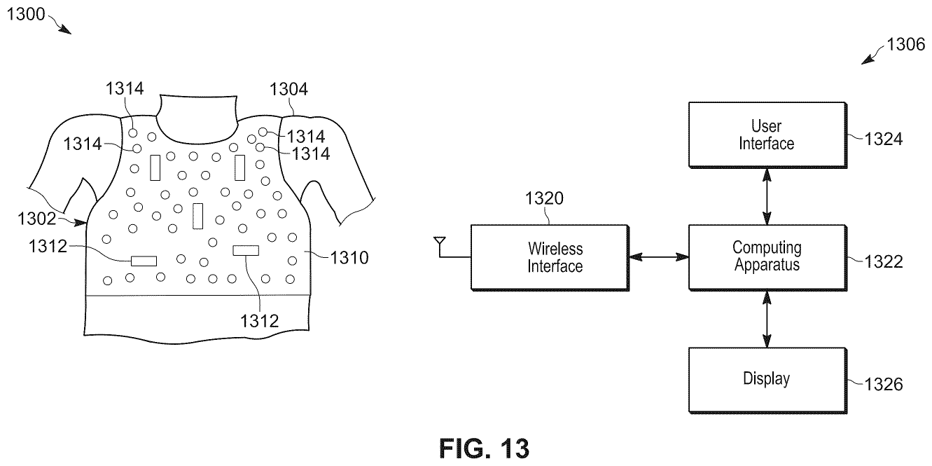

FIG. 13 illustrates an example sensing and analysis system 1300 including a sensing system 1302 applied to a patient's torso 1304. The system 1300 also includes a remote monitoring/analysis system 1306. The sensing system 1302 communicates with the remote system 1306 through a communication link, which in the example of FIG. 13 is a wireless communication link. As a result, the remote system 1306 can be at the same location as the sensing system 1302 or it can be located at a different location. That is, the wireless communication link enables a telemedicine approach in which the sensing apparatus is applied to the patient, which is located at a first location (e.g., a hospital, home or a clinic) and the remote system 1306 can be located at one or more other locations (e.g., another hospital, office, institution etc.).

The sensing system 1302 includes an arrangement of sensing apparatuses distributed across a substrate sheet 1310, such as the garment 1200 shown in FIG. 12 (e.g., a vest or other wearable apparatus). As described herein, the sensing apparatuses can include an arrangement of multimodal integrated sensing apparatuses 1312 (e.g., instances of sensing apparatus 102, 302, 502 or 1206), including an electrophysiological sensor, a transducer and associated circuitry, such as described herein. The sensing system 1302 can also include one or more other types of sensors 1314 also carried by the substrate sheet, such as can be electrophysiological sensors (e.g., electrodes). In other examples, all the sensors can be integrated multimodal sensing apparatuses, similar to the example sensing apparatus 1100 of FIG. 11.

FIG. 13 shows a back sensor section of the sensing system 1302 applied to the patient's torso. The apparatus thus is shown in the form of a vest, such as sensing apparatus 1200 shown in FIG. 12, and can include an arrangement of sensing apparatuses 1312, 1314 covering the patient's thorax, including the front, shoulders and sides of the patient's upper torso above the waist. In other examples, differently configured sections having a fewer or greater number of sensing apparatuses 1312, 1314 and/or a different distribution and/or density of sensing apparatuses can be implemented according to application requirements.

In one example, the remote system 1306 is configured to process physiological information received from the sensing apparatus through the wireless link, such as to generate one or more output visualizations based on the physiological information acquired by sensing apparatuses 1312, 1314 implemented in the sensing system 1302. One or more of the multimodal integrated sensing apparatuses 1312 can also include a wireless communication interface configured to communicate wirelessly with a wireless interface 1320 of the remote system 1306. For example, the wireless communication link can be implemented to include one or more wireless links implemented according to one or more wireless communication technologies, such as an 802.11x standard, Bluetooth, cellular (e.g., GSM, 4G, 5G) and/or another wireless communication technology. In some examples, the communication link between the sensing system 1302 and the remote system 1306 can include a network of wireless communication links between each of the respective integrated sensing apparatuses 1312 and the wireless interface 1320. In other examples, the integrated sensing apparatuses 1312 can be configured in a daisy-chain configuration or master-slave configuration. For instance, a selected one of the integrated sensing apparatuses 1312 is configured to communicate directly with the wireless interface 1320, and the other integrated sensing apparatuses 1312 communicate directly or indirectly with the selected module (e.g., through a wireless or physical communication medium).

In the example of FIG. 13, the remote system 1306 also includes a computing apparatus 1322, a user interface device 1324 and a display 1326. For example, the remote system 1306 can be implemented as an integrated unit (e.g., a cellular telephone, tablet computer, notebook computer or special purpose computing device) configured to process and analyze the physiological signals measured by the sensing apparatuses 1312, 1314. For example, computing apparatus 1322 can include a processor having instructions programmed to perform filtering, signal processing and analysis the physiological information (e.g., imaging data and electrophysiological signal measurements) received from the sensing apparatus by the wireless interface 1320 through a wireless link. In other examples, the remote system 1306 can be implemented as a set of discrete components in the form of a workstation.

As described herein, the sensing system 1302 can stream (e.g., via one or more wireless communication links) real-time ultrasound image data and electrophysiological data to the remote system 1306, and the computing apparatus 1322 can process such information to provide corresponding graphical output on display 1326 to visualize such information. The visualization can include a graphical representation of electrophysiological information mapped spatially onto an anatomical surface of interest (e.g., a three-dimensional or four-dimensional image of the patient's heart). Because the physiological information provided by the sensing system 1302 can include both image data and electrophysiological data, the computing apparatus 1322 can generate the visualization to include both mechanical (e.g., rendered as a 3D or 4D image) and electrophysiological information over a number of cardiac cycles, and can be synchronized in time. In some examples, given sufficient processing capabilities for the computing apparatus, the visualization can provide a near real-time or even real-time visualization of such multi-modal physiological information.

In an example, the system 1300 can be used to perform a physiological study based on a combination of ultrasound images, auscultation recordings and/or electrophysiological signals obtained concurrently over one or more time intervals. In another example, the system 1300 can be used to monitor a patient's physiological condition during an intervention (e.g., using manual and/or robotic techniques), such as ablation, cardiac resynchronization therapy, valve repair or replacement, and the like. The computing device 1322 can guide (e.g., through analysis and results provided on the display) and/or control the intervention based on a combination of ultrasound images, auscultation recordings and/or electrophysiological signals acquired concurrently over one or more time intervals. As mentioned above, the wireless communication link enables the user of the remote system to be either co-located in a common space with the patient and sensing system 1302, or the user can be at a remote (different) location from the patient. The remote location can be in a different room, a different building on a given campus, a different city or even a different country.

In other examples, the remote system can be implemented portable monitoring device (e.g., similar to a Holter monitor) such as for storing in non-transitory memory physiological signals measured by the sensing apparatuses 1312, 1314 over an extended period of time (e.g., a number of hours, days, weeks or more), which can be uploaded for further processing and/or analysis. As described herein, the physiological signals measured by the sensing apparatuses 1312, 1314 can include a combination of ultrasound images, auscultation recordings and/or electrophysiological signals obtained concurrently over time. The aggregate data that is acquired thus provides mechanical and electrical information to help determine or diagnose more complex conditions and comorbidities.

The display 1326 can be coupled to the computing apparatus 1322, and the user interface (e.g., a graphical user interface) 1324 can be associated with the computing apparatus 1322, such as for enabling a user to control the data acquisition process and to ensure that appropriate sensor connections have been made. The user interface can also be used to control operating parameters of ultrasound transducers in the multimodal sensing apparatuses 1312 for acquiring ultrasound images. The display 1326 may present the GUI to facilitate such controls. The computing apparatus 1322 can also be programmed to provide various features associated with the sensors and the data acquisition process. As an example, a user can employ a pointing device (e.g., a mouse or touch screen) or other input device (e.g., a keyboard or gesture control) to interact with the computing apparatus 1322. Such interactions can change the graphical and textual information on the display 1326. For instance, the user interface 1324 can be utilized to change between different sensor views or to enable interaction between multiple views that may be displayed concurrently for different parts of the system 1300.

As another example, a user can select one or more sensing apparatuses 1312, 1314 via the user interface 1324, such as can be presented on the display 1326 as part of an interactive graphical representation of a torso generated from the ultrasound images acquired by sensing apparatuses 1312. Several different interactive views of the sensing system 1302 can be provided, which can be utilized to configure and select the sensing apparatuses 1312, 1314.

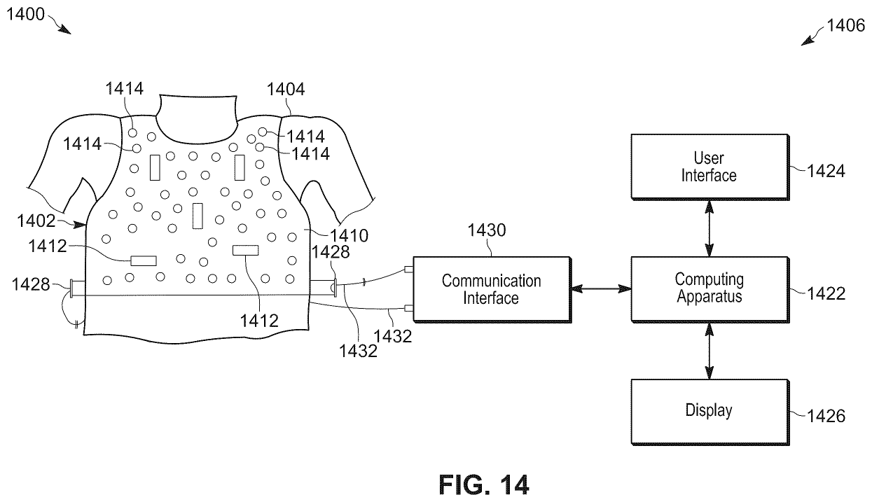

FIG. 14 illustrates an example sensing system 1400 including a sensing apparatus 1402 applied to a patient's torso 1404 and a remote monitoring/analysis system 1406. The system 1400 is similar to the system 1300 of FIG. 13 except in FIG. 14 the communication link includes a physical medium coupled between the sensing apparatus and the remote system 1406. Accordingly, the same reference numbers, increased by adding 100, are shown in FIG. 14 to refer to the same parts and features shown in FIG. 13.

In the example of FIG. 14, the sensing apparatus 1402 communicates with the remote system 1406 through a physical link. As a result, communication link can be configured to communicate power and information between the sensing apparatus 1402 and the remote system 1406. As shown, the sensing apparatus 1402 includes one or more connectors 1428 configured to be electrically connected to a communication interface 1430 through electrically conductive cables, schematically indicated at 1432. In one example, the cables 1432 from the sensor apparatus 1402 flow in a direction toward a respective side of the patient such as where the communication interface 1430 can be located. Each of the cables 1432 can provide a set of input signals to the communication interface 1430, and there can be any number of such cables depending on, for example, the configuration of the sensor apparatus 1402.

The communication interface 1430 can include amplifiers and other circuitry configured to receive and aggregate signals from respective sets of cables 1432 from different sensor circuits. The communication interface 1430 thus can be configured to amplify and filter (e.g., baseline noise removal filtering) the signals from each of the sensing apparatuses 1412, 1414 and provide a corresponding set of signals to the computing apparatus 1422. Additional filtering, signal processing and analysis can be implemented by the computing apparatus 1422.

As described herein, the sensing system 1402 can stream (e.g., via a communication link) real-time multi-modal physiological information (e.g., ultrasound image data, electrophysiological data, etc.) to the remote system 1406, and the computing apparatus 1422 can process such information to provide corresponding graphical output on display 1426 to visualize such information, which can provide a near real-time or even real-time visualization of such multi-modal physiological information, as described herein (e.g., with respect to FIG. 13).

In some examples, such as where the systems methods are used for electrocardiographic imaging, spatial geometry among the electrophysiological sensors and anatomy is needed. Typically, such geometry is determined from three-dimensional image data, such as acquired using computed tomography or magnetic resonance imaging modalities while a sensor apparatus is on the patient's body. However, such imaging modalities are expensive to use and may not be available in some settings. The sensing apparatuses, systems and methods disclosed herein can determine geometry information without using computed tomography or magnetic resonance imaging modalities and without using a spatial tracking system. This is enabled by use of ultrasound transducers integrated into the sensing system, as shown and described herein.

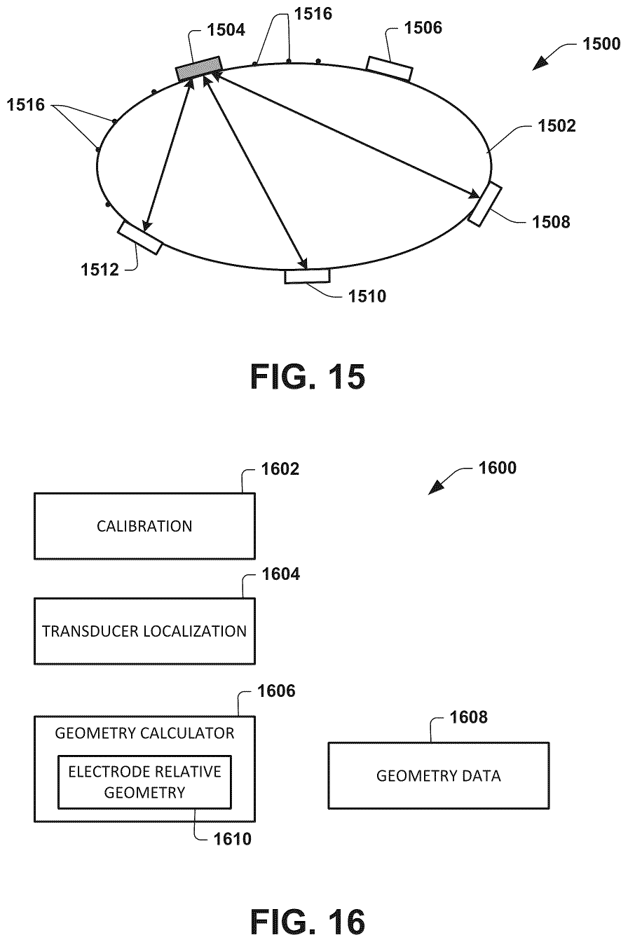

By way of example, FIG. 15 is a diagrammatic view of a cross section of part of a body (e.g., the thorax) 1502 to which a sensing apparatus has been placed. In the example of FIG. 15, the sensing apparatus includes an arrangement of integrated multimodal sensing apparatuses 1504, 1506, 1508, 1510 and 1512 at respective locations on the body 1502. Each of the multimodal sensing apparatuses 1504, 1506, 1508, 1510 and 1512 includes an ultrasound transducer and an electrode having a known fixed spatial relationship for each module. The number, location and density of ultrasound transducers implemented on the sensing apparatus can be configured to enable construction of a three-dimensional image volume for anatomy of interest, such as including the heart, lungs or other anatomy of interest.

FIG. 16 depicts an example workflow diagram 1600 for a method determining geometry data using a sensing apparatus. The method for determining geometry can be controlled by a remote system (e.g., system 1306 or 1406) and/or the method can be controlled by one or more transducer modules on the sensing apparatus. The following example will be described in the context of and with reference to FIG. 15; though, the method can be implemented with respect to any sensing apparatus that includes an arrangement of ultrasound transducers distributed across a flexible, conformable substrate that is applied to three-dimensional body.

As an example, the multimodal sensing apparatuses 1504, 1506, 1508, 1510 and 1512 can enter a calibration function 1602, during which operating parameters of each of the ultrasound transducers are tuned to enable formation of an image volume within the patient's body where the sensing apparatus is placed. In some examples, such as shown in FIG. 15, there can be communication of ultrasonic signals or other acoustic signals between respective pairs of transducers and/or respective individual image volumes can be generated and co-registered. The image volumes generated for calibration 1602 can be acquired over a fixed or variable time. The registration between image volumes can be implemented in an automated or semi-automated method, such as in response to a user input through a graphical user interface to select common points (e.g., pixels or voxels) within respective image volumes.

In a further example, which can be implemented as part of the calibration function 1602 or a transducer localization function 1604, distances between respective transducer modules can be determined. For example, each of the multimodal sensing apparatuses 1504, 1506, 1508, 1510 and 1512 can be configured to sequentially (or otherwise separately) transmit an ultrasonic signal and receive waves reflected ultrasonic signals. A distance to reflected structures, including other transducer modules, can be calculated based on the reflected from reflected ultrasonic signals (e.g., distance is proportional to the time and speed of the ultrasonic waves propagating in the body 1502). The distances can be determined between a given module and all other modules, and the process can be repeated for each of the respective modules relative to the other modules.

For example, the calibration function 1602 is configured to use an image segmentation function to identify the transducers in the ultrasound image volume, which can include an automated identification function and/or selection by a user input through a graphical user interface. For each identified transducer, the calibration function can determine the inter-transducer distance based on the time (e.g., time difference between transmit and receive times) and the known speed of the ultrasonic signals through the body. However, in some sensing apparatus configurations, some of the modules (e.g., multimodal sensing apparatus 1506) may not be located in the path of the transmitted ultrasonic signal from a transducer of a given multimodal sensing apparatuses (e.g., sensing apparatus 1504), and therefore unable to provide reflected signals to produce distance calculations between such modules (e.g., between transducers of apparatuses 1504 and 1506). The calibration function 1602 can be implemented for each of the multimodal sensing apparatuses 1504, 1506, 1508, 1510 and 1512, and the resulting computed distances for each module can be stored (e.g., in an array or other data structure) in memory.

The transducer localization function 1604 is configured to spatially localize each of the multimodal sensing apparatuses 1504, 1506, 1508, 1510 and 1512 in a three-dimensional coordinate system. In one example, the coordinate system can be determined relative to a selected one of the multimodal sensing apparatuses (e.g., apparatus 1504) based on the set of distance calculations. The localization function 1604 can use the computed distances between multimodal sensing apparatuses 1504, 1506, 1508, 1510 and 1512 to construct three-dimensional locations each of the modules relative to the selected module.

In another example, transducer localization function 1604 is configured to spatially localize the each of the multimodal sensing apparatuses 1504, 1506, 1508, 1510 and 1512 in a three-dimensional coordinate system based processing applied to ultrasound image volumes generated by each of the multimodal sensing apparatuses 1504, 1506, 1508, 1510 and 1512. For example, the localization function 1604 can be configured to stitch together the ultrasound image volumes produced by ultrasonic transducers of the multimodal sensing apparatuses 1504, 1506, 1508, 1510 and 1512 through an image registration process to provide a compounded three-dimensional image volume. The image registration can be automated and/or be guided in response to user input selection of common points in the respective image volumes generated by the transducer modules.

The localization function 1604 can be configured to perform a 3D reconstruction of points (e.g., pixels or voxels) in the compounded image volume. For example, the localization function can reconstruct by triangulation of points from multiple projection matrices determined for the image volume of each respective transducer module. The localization can implement a reconstruction method to reconstruct 3-dimensional points (in a common 3D coordinate system) based on a rectification transform that models each ultrasound transducer module as a stereo camera. An example of a triangulation method that can be implemented by function 1604 for stereo reproduction of a 3D image from 2D sources (respective ultrasound transducers) is disclosed in Y. Furukawa and C. Hernandez, Multi-View Stereo: A Tutorial, Foundations and Trends® in Computer Graphics and Vision, vol. 9, no. 1-2, pp. 1-148, 2013, which is incorporated herein by reference.

A geometry calculator 1606 is configured to determine geometry data 1608 representative of spatial geometry of the electrophysiological sensors (e.g., electrodes), ultrasound transducers and patient anatomy. For example, the geometry for each of the electrodes and ultrasound transducers can be represented as 3D spatial coordinates for a centroid of the respective electrodes and transducers. The geometry of the patient anatomy can be modeled as a set of 3D spatial coordinates defining the anatomical surface or as a model thereof (e.g., a mesh model), such as for the heart, lungs, brain or other anatomical structures of interest. In some examples, one or more of the anatomical models can be a 4D model that varies over time.

The geometry calculator 1606 can determine the geometry data based on the transducer locations (e.g., determined by transducer localization function 1604) and data describing known geometry of the electrodes relative to the transducers, shown at 1610. For example, the electrode relative geometry data 1610 can represent a 2D or 3D position of each electrode relative to a nearest one or more transducer module (in a coordinate system of the sensing apparatus). For electrodes that are integrated with respective transducer modules, the spatial coordinates of the associated (integrated) electrode can be derived from the transducer coordinates with a high level of accuracy. For electrodes or other sensors (e.g., 1002, 1204, 1314, 1414, or 1516) that are carried by the flexible substrate separately from being in an integrated transducer module, the relative location can similarly be determined for one or more transducer modules and stored in memory as the relative location data 1610. The relative location data 1610 thus can be used co-register the position of such electrodes or other sensors from the coordinate system of the sensing apparatus into the common spatial coordinate system with the patient anatomy and transducer modules. Appropriate anatomical landmarks or other fiducials, including locations for some or all the electrodes, can also be identified in the ultrasound image volume to enable registration of the electrode locations in the coordinate system. The identification of such landmarks can be done manually (e.g., by a person via image editing software) or automatically (e.g., via image processing techniques). As a result, the geometry data 1608 can be provided to represent geometry for each of the sensors and relevant patient anatomy in a common spatial domain.

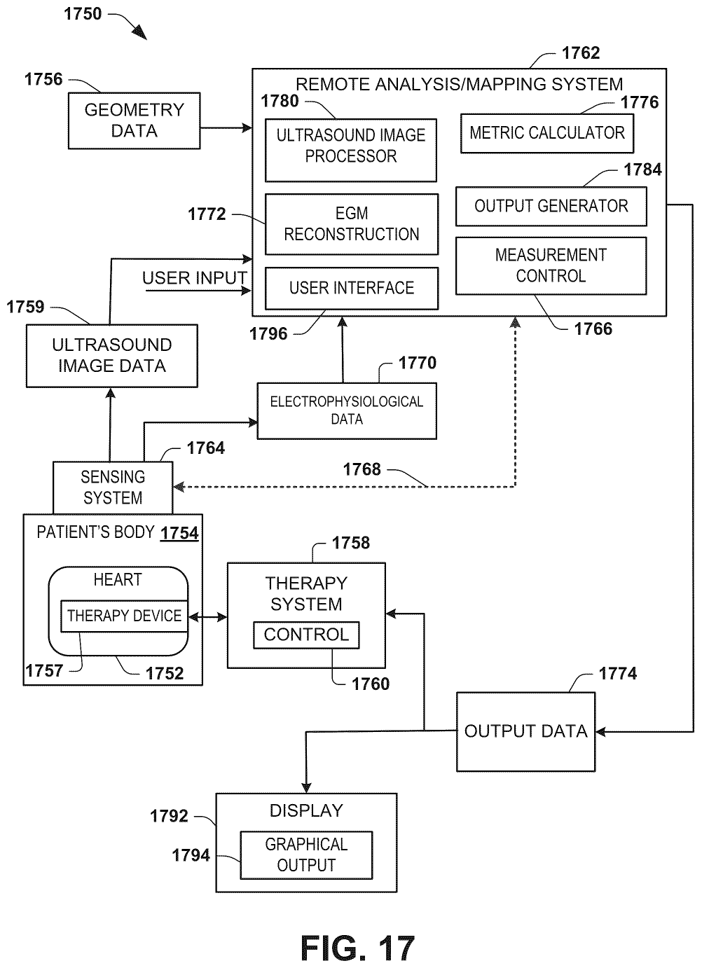

FIG. 17 depicts an example of a system 1750 that can be utilized for performing medical testing (diagnostics, screening and/or monitoring) and/or treatment of a patient. In some examples, the system 1750 can be implemented to generate one or more physiological maps based on physiological information obtained from a patient's body 1754. As described herein, the maps can include electrophysiological maps (e.g., electrocardiographic maps) generated for a patient's heart 1752 based on electrophysiological signals measured from the patient's body 1754 and geometry data 1756. Additionally or alternatively, the system 1750 can be utilized as part of a medical intervention, such as to help a physician determine parameters for delivering a therapy to the patient (e.g., delivery location, amount and type of therapy) or performing another type of intervention based on one or more electrocardiographic maps and/or other images that are generated based on physiological data acquired before and/or during the intervention. Other types of interventions can include surgical procedures, as such manual (e.g., handheld) surgery and/or robotic surgery techniques, which may include minimally invasive or invasive (e.g., open cavity) procedures.

As an example, a catheter or other probe having one or more interventional devices 1757 affixed thereto can be inserted into a patient's body 1754 as to contact the patient's heart 1752, endocardially or epicardially. In other examples, the device 1757 can be a non-contact probe configured to deliver therapy to the patient's heart or other tissue. The placement of the device 1757 can be guided based on geometry data 1756, information provided from one or more electroanatomic maps and/or from a 3D or 4D ultrasound image volume, such as can be generated by a remote analysis/mapping system 1762, as described herein. The guidance can be automated, semi-automated or be manually implemented based on physiological information acquired from the patient's body 1754. The functions of the remote system 1762 may be implemented as machine-readable instructions executable by one or more processors.

As a further example, an interventional system 1758 can be located external to the patient's body 1754 and be configured to control therapy or other intervention that is being provided through the device 1757. The interventional system 1758 can include controls (e.g., hardware and/or software) 1760 that can communicate (e.g., supply) electrical signals via a conductive link electrically connected between the intervention device (e.g., one or more electrodes) 1757 and the interventional system 1758. One or more sensors (not shown) can also communicate sensor information from the intervention device 1757 back to the interventional system 1758. The position of the device 1757 relative to the heart 1752 can be determined and tracked intraoperatively using real-time ultrasound imaging (e.g., by an arrangement of transducer modules that form part of a sensing system 1764. In some examples, a tracking modality can also be used to track and display location of the intervention device 1757. The location of the device 1757 and the therapy parameters thus can be combined to determine and control corresponding application of therapy.

Those skilled in the art will understand and appreciate various type and configurations of intervention devices 1757 that can be utilized, which can vary depending on the type of intervention. For instance, the device 1757 can be configured to deliver electrical therapy (e.g., radiofrequency ablation or electrical stimulation), chemical therapy, sound wave therapy, thermal therapy or any combination thereof. Other types of devices can also be delivered via interventional system 1758 and the invasive intervention device 1757 when positioned within the body 1754, such as to implant an object (e.g., a heart valve, stent, defibrillator, pacemaker or the like) and/or perform a repair procedure. The interventional system 1758 and intervention device 1757 may be omitted in some examples.

In the example of FIG. 17, a sensing system 1764 (e.g., sensing system 1000, 1100, 1200, 1302 1402) includes one or more integrated multimodal sensing apparatuses, such as described herein (e.g., sensing apparatus 102, 302, 502). For example, the sensing system 1764 includes an arrangement of multimodal sensing apparatuses distributed across one or more flexible and conformable sheet substrates (e.g., a wearable garment or patch). In some examples, because the resulting geometry data can be derived from ultrasound transducers, one or more multimodal sensing apparatuses can be individually mounted on a surface of the patient's body 1754 at desired locations. Each multimodal sensing apparatus can include an ultrasound or auscultation transducer and an electrophysiological sensor. In some examples, the sensing system 1764 also include a high-density arrangement of body surface electrophysiological sensors (e.g., greater than approximately 200 electrodes) that are distributed over a portion of the patient's torso spaced apart and separately from the multimodal sensing apparatuses. The electrophysiological sensors can be configured and arranged for measuring electrophysiological signals associated with the patient's heart (e.g., as part of an electrocardiographic mapping procedure). In another example, the sensing system 1764 can be implemented as a patch or panel that includes one or more multimodal sensing apparatuses on a flexible sheet substrate that does not cover the patient's entire torso, such as designed for measuring physiological information for a particular purpose (e.g., an array of electrodes and ultrasound transducers specially designed for measuring anatomical and electrophysiological signals associated with atrial fibrillation and/or ventricular fibrillation) and/or for monitoring physiological information for a predetermined spatial region of the heart (e.g., atrial region(s) or ventricular region(s)).

One or more electrophysiological sensors (e.g., electrodes) may also be located on the device 1757 that is inserted into the patient's body 1754. Such sensors can be utilized separately or in conjunction with the sensors in the non-invasive sensing system 1764 for mapping electrical activity for an endocardial surface, such as the wall of a heart chamber, as well as for an epicardial surface.

In each of such example approaches for acquiring patient physiological information, including acquiring ultrasound images, non-invasively sensing electrophysiological signals or a combination of invasive and non-invasive sensing electrophysiological signals, the sensing system 1764 provides electrophysiological data and ultrasound image data to the remote system 1762. In an example, the remote system 1762 includes measurement control 1766, which can be coupled to the sensing system 1764 through a communication link, shown as dotted line 1768. As described herein, the communication link can be a wireless link, a physical link or a combination of wireless and physical links. The sensing system 1764 thus can provide electrophysiological data 1770 and ultrasound image data 1759 based on respective physiological measurements that are performed, which can be stored in memory and communicated to the remote system 1762 through the communication link 1768. The electrophysiological data 1770 can include analog and/or digital information (e.g., representative of electrophysiological signals measured via electrodes). The ultrasound image data 1759 can be separate image volumes produced by respective ultrasound transducers or be a compounded image volume, such as described herein.

The control 1766 can be configured to control the data acquisition process (e.g., sample rate, line filtering) for measuring electrophysiological signals to provide the data 1770. The control 1766 can also be configured to control the image acquisition process (e.g., ultrasound mode, frequency gain or other parameters) for transmitting and receiving ultrasound signals to provide the data 1759. In some examples, the control 1766 can control acquisition of electrophysiological data 1770 separately from the ultrasound image data 1759, such as in response to a user input. In other examples, the electrophysiological data 1770 can be acquired concurrently with and in synchronization with ultrasound image data 1759. For example, appropriate time stamps can be utilized for indexing the temporal relationship between the respective data 1759 and 1770 as to facilitate the evaluation and analysis thereof by the remote system 1762.

The remote analysis/mapping system 1762 can also be programmed to perform other signal processing techniques on the physiological information (e.g., ultrasound image data 1759 and electrophysiological data 1770) received from the sensing system 1764. In an example, the remote analysis/mapping system 1762 can be programmed to apply a blind source separation (BSS) method on the set of electrophysiological signals represented by the electrophysiological data 1770. The BSS method is particularly useful extracting pertinent electrophysiological signals of interest measured by dry electrodes implemented in the sensing system 1764.

The remote system 1762 can further be programmed to combine electrophysiological data 1770 with geometry data 1756 by applying appropriate processing and computations to provide corresponding output data 1774. Additionally, in some examples, the remote system 1762 can also use the ultrasound image data 1759 to generate the output data 1774. The geometry data 1756 may correspond to ultrasound-based geometry data, such as be determined according to the example approach of FIG. 16. The geometry data 1756 thus represents a geometric relationship between points on one or more anatomical surface (e.g., a cardiac surface) and the sensors positioned on the torso surface in a three-dimensional coordinate system. As an example, the output data 1774 can include one or more graphical maps demonstrating determined electrophysiological signals reconstructed on a geometric surface representative of the patient's heart 1752 (e.g., information derived from electrophysiological measurements superimposed on a graphical representation of a surface of a heart).

The remote system 1762 can provide the output data 1774 to represent multimodal physiological information for one or more regions of interest or the entire heart in a temporally and spatially consistent manner. For example, the sensing system 1764 can measure electrophysiological signals and provide electrophysiological data 1770 for a predetermined region or the entire heart concurrently (e.g., where the sensing system 1764 covers the entire thorax of the patient's body 1754). The sensing system 1764 can also obtain spatially and temporally consistent ultrasound images for the same predetermined region or the entire heart. The electrical and mechanical information can be correlated over time to determine one or more physiological metrics, which can be provided as part of the output data 1774 (e.g., visualizing relationships between electrocardiographic maps derived from measured electrophysiological signals along with mechanical properties of the heart derived ultrasound images). The time interval for which the output data/maps are computed can be selected based on user input (e.g., selecting a timer interval from one or more waveforms). Additionally or alternatively, the selected intervals can be synchronized with the application of therapy by the interventional system 1758.

For example, the remote system 1762 includes an electrogram (EGM) reconstruction function 1772 programmed to compute an inverse solution and provide corresponding reconstructed electrograms based on the electrophysiological data 1770 and the geometry data 1756. The reconstructed electrograms thus can correspond to electrocardiographic activity across a cardiac envelope, and can include static (three-dimensional at a given instant in time) and/or be dynamic (e.g., four-dimensional map that varies over time). Examples of inverse algorithms that can be implemented by electrogram reconstruction 1772 include those disclosed in U.S. Pat. Nos. 7,983,743 and 6,772,004. The EGM reconstruction function 1772 thus can reconstruct the body surface electrophysiological signals measured via electrodes of the sensing system 1764 onto a multitude of locations on a cardiac envelope (e.g., greater than 1000 locations, such as about 2000 locations or more

As disclosed herein, the cardiac envelope can correspond to a 3D surface geometry corresponding to the heart, which surface can be epicardial and/or endocardial surface model derived at least in part from ultrasound image data. For example, the locations may be nodes distributed across a mesh model (e.g., corresponding to the points defined by cardiac surface data 230, 1730) derived from ultrasound image data 1759, as disclosed herein. The locations of the nodes in the mesh model can be static (e.g., 3D points) or dynamic (e.g., 4D locations that vary over time), such as derived from a set of the ultrasound image data 1759.

As mentioned above, the geometry data 1756 can correspond to a mathematical model that has been constructed based on ultrasound image data for the patient. Thus, the geometry data 1756 that is utilized by the electrogram reconstruction function 1772 can correspond to actual patient anatomical geometry. In another example, the geometry data can include a preprogrammed generic model or a combination of patient anatomy and a generic model (e.g., a model/template that is modified based on patient anatomy). By way of further example, the ultrasound imaging and generation of the geometry data 1756 may be performed concurrently with recording the electrophysiological signals that is utilized to generate the electrophysiological data 1770. In another example, the ultrasound imaging can be performed separately (e.g., before or after the measurement data has been acquired) from the electrical measurements.

Following (or concurrently with) determining electrical potential data (e.g., electrogram data computed from non-invasively or from both non-invasively and invasively acquired measurements) across the geometric surface of the heart 1752, the electrogram data can undergo further processing by remote system 1762 to generate the output data 1774. The output data 1774 may include one or more graphical maps of electrophysiological signals or information derived from such signals.

An output generator 1784 can be programmed to generate the output data 1774 based on one or more of the electrophysiological data 1770, the ultrasound image data 1759, processed ultrasound data (derived by an ultrasound image processor 1780) and/or reconstructed electrophysiological signals (computed by EGM reconstruction function 1772). The remote system 1762 can provide the output data 1774 to one or more displays 1792 to provide a visualization including include one or more graphical outputs 1794 (e.g., waveforms, electroanatomic maps, related guidance, or the like). The remote system 1762 can also include a metric calculator 1776 having one or more computation methods programmed to characterize the physiological information for the patient based on one or more of the electrophysiological data 1770, the ultrasound image data 1759, processed ultrasound data (derived by an ultrasound image processor 1780) and/or reconstructed electrophysiological signals (computed by EGM reconstruction function 1772). The metric calculator can also characterize physiological information derived from acoustic waves received by the transducer (e.g., auscultation transducer) into data representative of physiological sounds of the heart, lungs (e.g., in the audible frequency range of about 10 Hz to about 20 KHz). Additionally, or alternatively, the acoustic waves received by the auscultation transducer can be amplified and supplied to an audio speaker for listening by one or more users.

The remote system 1762 can also include a user interface (e.g., a graphical user interface) 1796 configured to control functions applied by the remote system 1762 and resulting output 1794 that is provided to the display 1792 in response a user input. For example, parameters associated with the displayed graphical output, corresponding to an output visualization of a computed map or waveform, such as including selecting a time interval, temporal and spatial thresholds, as well as the type of information and/or viewing angle that is to be presented in the display 1792 can be selected in response to a user input via the user interface 1796. For example, a user can employ the GUI 1796 to selectively program one or more parameters (e.g., temporal and spatial thresholds, filter parameters, metric computations, and the like) used by the one or more functions 1772, 1776, 1780 to process the ultrasound image data 1759, electrophysiological data 1770 and geometry data 1756. The remote system 1762 thus can generate corresponding output data 1774 that can in turn be rendered as a corresponding graphical output in a display 1792, such as including one or more graphical output visualizations 1794. For example, the output generator 1784 can generate graphical maps and other output visualizations, which can be superimposed on an anatomical model or on a 3D or 4D ultrasound image (e.g., a real-time or prerecorded image) based on the ultrasound image data 1759.

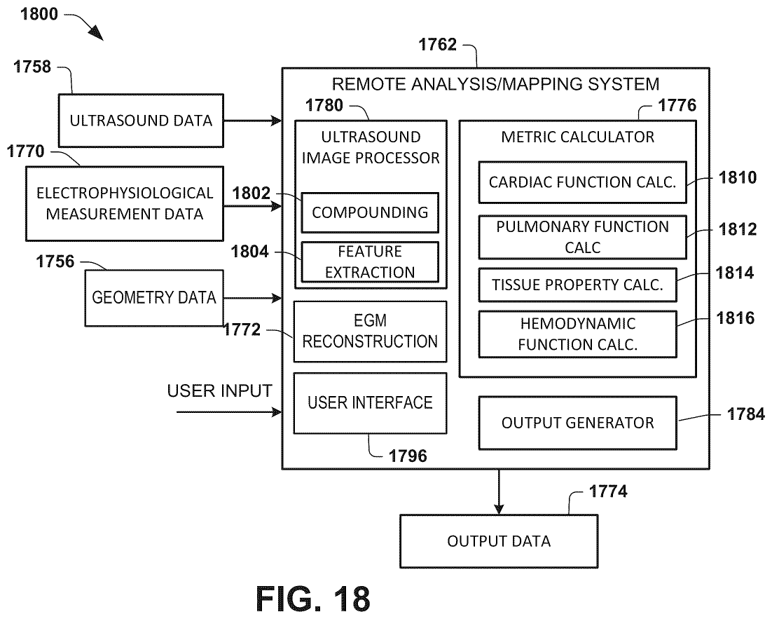

FIG. 18 is a block diagram 1800 showing an example of some functions that can be implemented by the remote analysis/mapping system 1762 of FIG. 17. Accordingly, the description of FIG. 18 also refers to FIG. 17.

In the example of FIG. 18, the ultrasound image processor 1780 includes compounding and feature extraction functions 1802 and 1804, respectively. The compounding function 1802 is programmed to perform spatial compounding of ultrasound images produced by the ultrasound transducer modules distributed across the patient's body. The spatial compounding function 1802 accesses the ultrasound image data (from memory) that have been generated the respective ultrasound transducer modules at different viewing angles over time (e.g., a number of image frames). The spatial compounding function 1802 also uses image registration to align the respective images. For example, the image registration function can be programmed to implement include intensity-based registration and/or fiducial-based registration. The image registration can be automated and/or be responsive to a user input selecting features from the respective images to be compounded. In some examples, the component images can be registered to a reference image, such as a prior 3D image volume of the anatomy acquired using another imaging modality (e.g., CT, MRI, or the like). The alignment of features among the respective images can also be based on computing a similarity or difference metric for the respective region represented by the respective images. As mentioned, anatomical landmarks or other fiducials (e.g., respective sensors and/or transducer modules) can be used for alignment among respective images. The landmarks and/or fiducials can be extracted (e.g., by invoking feature extraction function 1804) or the landmarks and/or fiducials can be selected through the user interface 1796 in response to a user input selecting the landmarks and/or fiducials. After the respective pixels or voxels of the respective ultrasound images have been aligned, the compounding function can construct a compounded image volume. The compounded image volume can be provided as a single image frame or as 4D image volume that varies over time. Advantageously, the compounding of the respective images can enhance the anatomical features (particularly in overlapping areas) as well as reduce speckle artifacts and other noise that might be present in the respective images prior to compounding.

The feature extraction function 1804 can be programmed to extract one or more features from the compounded image volume. The feature extraction function 1804 can be applied automatically, such in response to function calls by one or more functions of the metric calculator 1776. In other examples, the feature extraction function 1804 can operate in response to a user input instruction specifying one or more features through the user interface 1796. For example, the feature extraction function 1804 can identify one or more anatomical surfaces (e.g., epicardial, endocardial, pulmonary surfaces or the like) or other objects (e.g., sensors, transducer modules, and the like) visible within the compounded image volume. The pixels or voxels for the extracted surfaces can be tagged and corresponding spatial coordinates of the surfaces can be stored in memory, such by specifying points on the surface or constructing a model. In some examples, the ultrasound image processor 1780 is programmed to generate the geometry data 1756, such as described herein, based on the compounded image volume. As a result, the spatial coordinates for the extracted anatomical surface or other objects can be provided in the same coordinate spatial coordinate system as the geometry data 1756.

As mentioned, the metric calculator 1776 is programmed to compute one or more metrics (e.g., quantitative assessments) for a number physiological conditions. In the example of FIG. 18, the metric calculator 1776 includes a cardiac function calculator 1810, a pulmonary function calculator 1812, a tissue property calculator 1814, and a hemodynamic functional calculator 1816. The metric calculator 1776 as well as each of its respective calculators 1810-1816 can compute such metrics based on one or more of the electrophysiological data 1770, the ultrasound image data 1759, processed ultrasound data (derived by an ultrasound image processor 1780) and/or reconstructed electrophysiological signals (computed by EGM reconstruction function 1772). The cardiac function calculator 1810 can be configured to analyze one or more frame of ultrasound images and/or electrophysiological information (e.g., measurement data 1770 and/or reconstructed electrophysiological signals on a cardiac surface) to compute a value representative of a cardiac function metric.

In one example, the cardiac function calculator 1810 is programmed to determine one or more anatomical mechanical properties based on analysis of the ultrasound image data 1759 and/or electrical properties based on reconstructed electrophysiological signals. For example, the cardiac function calculator 1810 can invoke the ultrasound image processor to segment the image and analyze dimensions of the heart and/or one or more of its chambers in one or more image frames acquired over time. Based on such analysis over a plurality of frames (including at least one full cardiac cycle), the cardiac function calculator 1810 can quantify one or more functional parameters, such as heart rate, stroke volume, cardiac output, and ejection fraction.

As a further example, the control 1766 can provide instructions to a selected one or more of the ultrasound transducer modules to operate the ultrasound in the B-mode and acquire respective images of cardiac anatomy, including long and short-axis views. The ultrasound image processor 1780 can analyze the acquired B-mode ultrasound images to determine spatial coordinates for epicardial and endocardial surfaces, including an identification of long and short axes of the heart. The cardiac function calculator 1810 (or other function) can determine a measure of wall thickness across the heart (e.g., distance between coordinates along the epicardial and endocardial surfaces. The cardiac function calculator 1810 can also determine stroke volume, ejection fraction, cardiac output, endocardial and epicardial area based on such measurements.

In another example, the control 1766 can provide instructions to a selected one or more of the ultrasound transducer modules to operate the ultrasound in the M-mode and acquire respective images of cardiac anatomy. The ultrasound image processor 1780 can analyze the acquired M-mode ultrasound images using feature extraction (e.g., automated and/or manually responsive to a user input selection of features) identify anatomical features of interest, such as one or more heart valves or other tissue. The cardiac function calculator 1810 can monitor motion of the identified features and track motion over time, such as to provide an assessment of valve plane motion and/or leaflet dynamics.

The pulmonary function calculator 1812 is programmed to determine one or more anatomical mechanical properties of pulmonary system (e.g., lungs) based on analysis of the ultrasound image data 1759. The pulmonary function calculator 1812 can use function of the ultrasound image processor 1780 in a similar manner to as described above with respect to the cardiac function calculator 1810. For example, the pulmonary function calculator 1812 can invoke the ultrasound image processor 1780 and its functions to segment and extract pulmonary structural features (e.g., representative of anatomical surfaces and/or fluid within spaces between surfaces) from one or a series of ultrasound images. The pulmonary function calculator 1812 can compute one or more pulmonary properties based on the extracted features, such as pneumothorax, pleural effusion, pneumonia/consolidation, volume assessment, such as representing volume changes (e.g., free fluid within the lungs).

The tissue property calculator 1814 is programmed to determine one or more properties of tissue (e.g., tissue properties of the heart, lung and other tissue) based on analysis of the ultrasound image data 1759 and/or electrophysiological data 1170. Examples of mechanical tissue properties that the tissue property calculator 1814 can determine based on ultrasound image data 1759 include strain, deformation, stiffness, and elasticity to name a few. Examples of electrical tissue properties that the tissue property calculator 1814 can determine based on ultrasound image data 1759 and/or electrophysiological data 1170 include impedance or conductivity.

For example, the ultrasound transducer modules and ultrasound image processor 1780 can be configured (e.g., by control 1766) to implement speckle tracking of cardiac tissue. The tissue property calculator 1814 can be programmed to compute and/or strain rates (e.g., global longitudinal strain, global circumferential strain, radial strain etc.) of respective tissue regions. The strain-related information can be used (e.g., by cardiac function calculator 1810) to provide a quantitative assessment of cardiac function for respective regions based on the determined strain properties. In some examples, the tissue property calculator 1814 can be programmed to provide a quantitative assessment of tissue stiffness, such as can be measured/inferred from ultrasound elastography measures (e.g., by computing a value representative of Young's modulus for tissue) based on tissue displacement (e.g., longitudinal deformation) determined responsive to ultrasonic signals or other acoustic energy transmitted by one or more of the transducer modules. The tissue property calculator 1814 can also be programmed to calculate an elastic modulus of tissue (e.g., stiffness of cardiac or lung tissue) can also be calculated using ultrasound shear wave velocity measurements and based on an estimated tissue density.

The hemodynamic function calculator 1816 is programmed to determine one or more fluid dynamic properties (e.g., of blood or other fluids present within the patient's body) based on analysis of the ultrasound image data 1759. For example, the hemodynamic function calculator 1816 is programmed to compute velocity gradient of blood based on speckle tracking methods (e.g., speckle decorrelation- and correlation-based lateral speckle-tracking methods performed by the ultrasound transducer processor). As a further example, the ultrasound image processor 1780 can process the acquired ultrasound images acquired over time to and analyze the pixels (or voxels) acquired through intermittent sampling over time and determine properties representative of fluid flow velocity and direction. For example, the ultrasound image processor 1780 can be programmed to implement color-flow Doppler in which flow (e.g., blood flow) having a positive or negative Doppler shifts are mapped to respective different color-codes depending on the directions of flow. The color-coded pixels can be rendered on a grey-scale or other (e.g., M-mode) image of the anatomy. The intensity or contrast of the respective colors can also be adjusted within a given color palate according to the velocity of the blood that is computed based on the change in pixel (or voxel) positions over time. The hemodynamic function calculator 1816 can compute properties that provide measure of blood velocity based on the velocity values of pixels within hollow portions of the tissue (e.g., heart chambers, blood vessels, lungs and other spaces).

In some examples, the metric calculator 1776 can be configured to instruct the ultrasound transducer modules and ultrasound image processor 1780 to implement other forms Doppler ultrasound or speckle tracking for computing one or more other metrics. For example, the ultrasound transducer modules and ultrasound image processor 1780 can implement power Doppler ultrasound can be implemented in which the amplitude of the Doppler signal is mapped to a continuous color range. Such power Doppler can be used to spatially identify small anatomical structures, such as blood vessels or calcified regions. In some examples, ultrasound contrast agents (injectable microspheres) can be injected into the patient's body (e.g., into the blood stream) to facilitate detection of blood flow dynamics in particular regions.

Any of the computed metrics 1776, 1810, 1812, 1814 and 1816 and associated graphical outputs thereof can be synchronized with respect to the measured electrophysiological signals, such as provided in one or more maps generated by the EGM reconstruction function 1772. For example, a graphical representation of the cardiac function information can be superimposed on a graphical representation of the electrocardiographic maps that is provided in a given window of the display 1792. In another example, the graphical representation of the cardiac function can be superimposed on an ultrasound image in a respective window of the display 1792 and the graphical representation of the electrocardiographic map can be displayed concurrently in a separate window of the display 1792. The electrophysiological information derived from the electrophysiological data and the mechanical information derived from the ultrasound data thus can be combined in various ways to provide complementary data for assessing the patient's condition.

As a further example, the remote system 1762 can generate the output data 1774 to provide guidance or controls based on the ultrasound image data 1759, electrophysiological data 1770 and associated maps, and/or one or more of the metrics computed by the metric calculator 1776. The guidance can be provided before, during (e.g., in real-time) or after an intervention. In some examples, the guidance and/or controls can be provided automatically based on applying rules (e.g., programmed responsive to a user input) to the ultrasound image data 1759, electrophysiological data 1770 and associated maps, and/or one or more of the metrics computed by the metric calculator 1776. The guidance can be presented in an output graphical visualization, and controls can be in the form of control applied to delivery of one or more therapy or other intervention. In yet another example, the guidance can be provided a robotically controlled surgical system based on which the robotically controlled (or computer-assisted) surgical system can control one or more parameters for moving one or more instruments other control functions as part of performing the intervention.

Referring back to FIG. 17, in some examples, the output data 1774 can include control instructions used by the interventional system 1758 or a user can adjust a therapy based on the graphical output 1794 on the display 1792. For example, the therapy control 1760 can be configured implement fully automated control, semi-automated control (partially automated and responsive to a user input) or manual control based on the output data 1774. As an example, the control 1760 can set one or more parameters to control delivery of ablation therapy (e.g., radiofrequency ablation, pulsed field ablation, cryoablation, etc.) to a region of the heart based on the output data identifying one or more arrhythmia drivers on a surface having a thickness exceeding a threshold. In other examples, an individual can view the graphical output (e.g., including real-time ultrasound images and electrocardiographic maps) 1794 on the display 1792 to manually control one parameters of the interventional system (e.g., location, type and level of therapy etc.). Other types of therapy and devices can also be controlled based on the output data 1774 and corresponding graphical map 1794 presented on the display 1792.

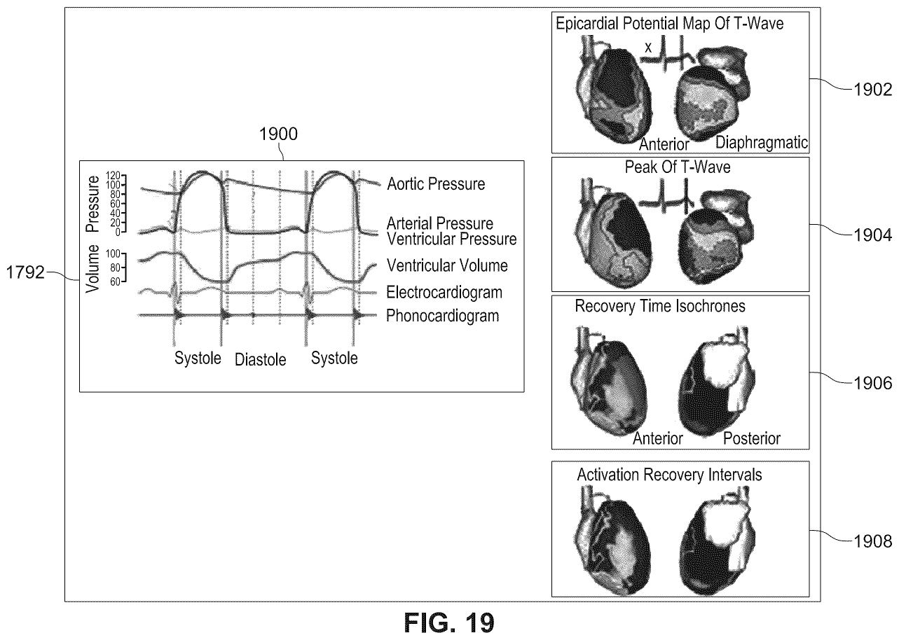

FIG. 19 depicts an example multi-modal outputs that can be generated on display 1792, such as by the system 1700 of FIG. 17 or 18. For example, the display 1792 includes a graphical visualization (e.g., a 2D image) 1900 of graphs visualizing different physiological information as a function of time, such as can be determined from data produced by the sensing system 1764 over the progression or one or more cardiac cycles. As described herein, the sensing system 1764 can include a single sensing device having an arrangement of multi-modal sensors configured to collect physiological information from the patient's body, such as including images, acoustic measurements and electrophysiological signals. In the example of FIG. 19, the physiological information in the visualization 1900 includes graphs of aortic pressure, arterial pressure, ventricular pressure, electrocardiogram and phonocardiogram over the progression of a cardiac cycle. FIG. 19 also includes a plurality of graphical (e.g., ECGI) maps 1902, 1904, 1906 and 1908 shown on a cardiac surface. The type of information presented in a respective map 1902, 1904, 1906 and 1908 can be selected in response to a user input, such as to provide electrophysiological information across the 3D surface which can be static information or be 4D information that varies over time. In some examples, the information provided in the 2D image 1900 and on one or more of the graphical maps 1902, 1904, 1906 and 1908 can present a diverse set of physiological information that is aligned (synchronized in time) over the progression of one or more cardiac cycles. Additionally, or alternatively, the information in the 2D image 1900 and/or one or more of the graphical maps 1902, 1904, 1906 and 1908 can represent respective physiological information over a user-selected time interval or provide real-time visualization of such information.

As described herein, because the systems and methods disclosed herein are configured to obtain and analyze multimodal physiological information, such as based on at least electrophysiological data and ultrasound image data obtained concurrently from a given patient, the resulting output data can provide broader, complementary and more encompassing assessment between electrophysiological conditions and biomechanical conditions (e.g., cardiac function, pulmonary function, hemodynamics, etc.) compared to existing systems.

The invention may be further described with respect to the following numbered paragraphs:
1. A system, comprising:
   a sheet flexible material having a contact surface adapted to be placed on an outer surface of a patient's body;
   a plurality of sensing apparatuses having respective sensing surfaces distributed across the contact surface of the sheet, at least one of the sensing apparatuses comprising:
   a multimodal sensing apparatus comprising:
      a transducer configured to at least sense acoustic energy from a transducer location of the sheet;
      circuitry coupled to the transducer;
      an electrophysiological sensor coupled to the circuitry, the sensor configured to at least sense electrophysiological signals from a sensor location of the sheet, in which the sensor location has a known spatial position relative to the transducer location; and
      a monolithic substrate carrying the transducer, the circuitry and the electrophysiological sensor.
2. The system of paragraph 1, wherein the monolithic substrate comprises one of a printed circuit board material or a packaging material.
3. The system of paragraph 1, wherein the transducer comprises a plurality of micromachined electromechanical systems (MEMS) transducer elements integrated on a respective integrated circuit die, the respective die including at least a portion of the circuitry carried by the substrate.
4. The system of paragraph 1, wherein:
   the transducer comprises an ultrasound transducer configured receive ultrasonic vibrations and convert the received ultrasonic vibrations to electrical signals, the circuitry on the respective die configured to process the electrical signals and provide ultrasound image data based on the received ultrasonic vibrations, and
   the electrophysiological sensor comprises an electrode.
5. The system of paragraph 1, wherein the transducer comprises an auscultation transducer configured to convert at least some of the received acoustic waves into electrical signals representative of audible sound.
6. The system of paragraph 1, wherein the sheet and the plurality of sensing apparatuses define a sensing system, the system further comprising:
   a remote system comprising:
   a communication interface configured to communicate with sensing system through a communication link; and
   a computing apparatus coupled to the communication interface, the computing apparatus configured to process information received from the at least one sensing apparatus through the communication link.
7. The system of paragraph 6, wherein the plurality of sensing apparatuses comprises respective instances of the multimodal sensing apparatus, in which the transducer of each of the respective instances comprises an ultrasound transducer configured receive ultrasonic vibrations and convert the received ultrasonic vibrations to corresponding electrical signals, wherein the circuitry on at least some of the respective instances of the multimodal module is configured to provide ultrasound image data based on the corresponding electrical signals, wherein the ultrasound image data includes ultrasound image frames of patient anatomy and at least some of the sensing apparatuses.
8. The system of paragraph 7, wherein the computing apparatus is configured to receive the ultrasound data through the communication link, and
   generate a compounded three-dimensional image volume representative of patient anatomy and locations of the at least some of the sensing apparatuses based on the ultrasound image data received from the sensing system.
The system of paragraph 8, wherein the computing apparatus is configured to determine locations of the plurality of electrophysiological sensors and at least one anatomical surface within the patient's body based on image processing of the three-dimensional image volume and the sensor position for each electrophysiological sensor known relative to the transducer location for each respective instance of the multimodal sensing apparatus, and
   provide geometry data representing a spatial relationship between the electrophysiological sensors and the anatomical surface in a three-dimensional coordinate system.
10. The system of paragraph 9, wherein the information received by the computing apparatus through the communication link further comprises electrophysiological data representative of electrophysiological signals measured by the respective electrophysiological sensors over time,
   wherein the computing apparatus is further configured to reconstruct electrophysiological signals on the anatomical surface based on the electrophysiological data and the geometry data.
11. The system of paragraph 10, wherein the computing apparatus is further configured to generate output data to visualize at least one of the compounded three-dimensional image volume and the reconstructed electrophysiological signals, and
   wherein the remote system further comprises a display configured to present a visualization based on the output data.
12. The system of paragraph 10, wherein:
   the anatomical surface comprises a cardiac envelope, and the electrophysiological signals are representative of cardiac electrophysiological signals measured by the respective electrophysiological sensors, and the visualization presented by the display includes real-time ultrasound image and a graphical representation of the reconstructed electrophysiological signals over time.
13. The system of paragraph 12, wherein the computing apparatus is further configured to calculate a metric that characterizes physiological information for the patient based on one or more of the electrophysiological data, the compounded three-dimensional image volume and the reconstructed electrophysiological signals.
14. The system of paragraph 13, wherein the metric comprises:
   a cardiac function calculator programmed to determine one or more anatomical mechanical properties based on analysis of the compounded three-dimensional image volume and/or electrical properties based on reconstructed electrophysiological signals;
   a pulmonary function calculator programmed to determine one or more anatomical mechanical properties of pulmonary system based on analysis of the compounded three-dimensional image volume;
   a tissue property calculator programmed to determine one or more properties of tissue based on analysis of the compounded three-dimensional image volume and/or the electrophysiological data; and/or
   hemodynamic function calculator programmed to determine one or more fluid dynamic properties based on analysis of compounded three-dimensional image volume.
15. The system of paragraph 7, further comprising an interventional system comprising a device configured to perform an intervention at a site within the patient's body, and wherein the computing apparatus is configured to provide guidance associated with the intervention being provided based on one or more of the electrophysiological data and the compounded three-dimensional image volume.
16. The system of paragraph 15, wherein the interventional system comprises a controller configured to control at least one parameter of the intervention based on the guidance.
17. The system of paragraph 1, wherein:
   each of the plurality of sensing apparatuses comprises an instance of the multimodal sensing apparatus, and the respective instances of the multimodal sensing apparatus are distributed across the sheet at respective sensing locations, or
the plurality of sensing apparatuses comprises:
   a number of instances of the multimodal sensing apparatus distributed across the sheet at respective locations; and
   a plurality of electrophysiological sensors distributed across the sheet at respective locations spaced from the instances of the multimodal sensing apparatus.

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a medical device.

In one or more examples, the described techniques may be implemented in hardware, software, firmware, or any combination thereof. If implemented in software, the functions may be stored as one or more instructions or code on a computer-readable medium and executed by a hardware-based processing unit. Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor” as used herein may refer to any of the foregoing structure or any other physical structure suitable for implementation of the described techniques. Also, the techniques could be fully implemented in one or more circuits or logic elements.

What is claimed is:

1. A system, comprising:
a sheet flexible material having a contact surface adapted to be placed on an outer surface of a patient's body;
a plurality of sensing apparatuses having respective sensing surfaces distributed across the contact surface of the sheet, at least one of the sensing apparatuses comprising:
a multimodal sensing apparatus comprising:
a transducer configured to at least sense acoustic energy from a transducer location of the sheet;
circuitry coupled to the transducer;
an electrophysiological sensor coupled to the circuitry, the sensor configured to at least sense electrophysiological signals from a sensor location of the sheet, in which the sensor location has a known spatial position relative to the transducer location; and
a monolithic substrate carrying the transducer, the circuitry and the electrophysiological sensor.

2. The system of claim 1, wherein the monolithic substrate comprises one of a printed circuit board material or a packaging material.

3. The system of claim 1, wherein the transducer comprises a plurality of micromachined electromechanical systems (MEMS) transducer elements integrated on a respective integrated circuit die, the respective die including at least a portion of the circuitry carried by the substrate.

4. The system of claim 1, wherein:
the transducer comprises an ultrasound transducer configured receive ultrasonic vibrations and convert the received ultrasonic vibrations to electrical signals, the circuitry on the respective die configured to process the electrical signals and provide ultrasound image data based on the received ultrasonic vibrations, and
the electrophysiological sensor comprises an electrode.

5. The system of claim 1, wherein the transducer comprises an auscultation transducer configured to convert at least some of the received acoustic waves into electrical signals representative of audible sound.

6. The system of claim 1, wherein the sheet and the plurality of sensing apparatuses define a sensing system, the system further comprising:
a remote system comprising:
a communication interface configured to communicate with sensing system through a communication link; and
a computing apparatus coupled to the communication interface, the computing apparatus configured to process information received from the at least one sensing apparatus through the communication link.

7. The system of claim 6, wherein the plurality of sensing apparatuses comprises respective instances of the multimodal sensing apparatus, in which the transducer of each of the respective instances comprises an ultrasound transducer configured receive ultrasonic vibrations and convert the received ultrasonic vibrations to corresponding electrical signals, wherein the circuitry on at least some of the respective instances of the multimodal module is configured to provide ultrasound image data based on the corresponding electrical signals, wherein the ultrasound image data includes ultrasound image frames of patient anatomy and at least some of the sensing apparatuses.

8. The system of claim 7, wherein the computing apparatus is configured to
receive the ultrasound data through the communication link, and
generate a compounded three-dimensional image volume representative of patient anatomy and locations of the at least some of the sensing apparatuses based on the ultrasound image data received from the sensing system.

9. The system of claim 8, wherein the computing apparatus is configured to
determine locations of the plurality of electrophysiological sensors and at least one anatomical surface within the patient's body based on image processing of the three-dimensional image volume and the sensor position for each electrophysiological sensor known relative to the transducer location for each respective instance of the multimodal sensing apparatus, and
provide geometry data representing a spatial relationship between the electrophysiological sensors and the anatomical surface in a three-dimensional coordinate system.

10. The system of claim 9, wherein the information received by the computing apparatus through the communication link further comprises electrophysiological data representative of electrophysiological signals measured by the respective electrophysiological sensors over time,
wherein the computing apparatus is further configured to reconstruct electrophysiological signals on the anatomical surface based on the electrophysiological data and the geometry data.

11. The system of claim 10, wherein the computing apparatus is further configured to generate output data to visualize at least one of the compounded three-dimensional image volume and the reconstructed electrophysiological signals, and
wherein the remote system further comprises a display configured to present a visualization based on the output data.

12. The system of claim 10, wherein:
the anatomical surface comprises a cardiac envelope, and the electrophysiological signals are representative of cardiac electrophysiological signals measured by the respective electrophysiological sensors, and
the visualization presented by the display includes real-time ultrasound image and a graphical representation of the reconstructed electrophysiological signals over time.

13. The system of claim 12, wherein the computing apparatus is further configured to calculate a metric that characterizes physiological information for the patient based on one or more of the electrophysiological data, the compounded three-dimensional image volume and the reconstructed electrophysiological signals.

14. The system of claim 13, wherein the metric comprises:
a cardiac function calculator programmed to determine one or more anatomical mechanical properties based on analysis of the compounded three-dimensional image volume and/or electrical properties based on reconstructed electrophysiological signals;
a pulmonary function calculator programmed to determine one or more anatomical mechanical properties of pulmonary system based on analysis of the compounded three-dimensional image volume;
a tissue property calculator programmed to determine one or more properties of tissue based on analysis of the compounded three-dimensional image volume and/or the electrophysiological data; and/or
hemodynamic function calculator programmed to determine one or more fluid dynamic properties based on analysis of compounded three-dimensional image volume.

15. The system of claim 7, further comprising an interventional system comprising a device configured to perform an intervention at a site within the patient's body, and
wherein the computing apparatus is configured to provide guidance associated with the intervention being provided based on one or more of the electrophysiological data and the compounded three-dimensional image volume.

16. The system of claim 15, wherein the interventional system comprises a controller configured to control at least one parameter of the intervention based on the guidance.

17. The system of claim 1, wherein:
each of the plurality of sensing apparatuses comprises an instance of the multimodal sensing apparatus, and the respective instances of the multimodal sensing apparatus are distributed across the sheet at respective sensing locations, or
the plurality of sensing apparatuses comprises:
a number of instances of the multimodal sensing apparatus distributed across the sheet at respective locations; and
a plurality of electrophysiological sensors distributed across the sheet at respective locations spaced from the instances of the multimodal sensing apparatus.

18. A system comprising:
a sensing system comprising;
an arrangement of electrophysiological sensors and ultrasound transducer modules on a flexible sheet adapted to be placed on an outer surface of a patient's body, the electrophysiological sensors configured to measure electrophysiological signals from the body surface, and the ultrasound transducer modules configured to measure acoustic waves from the body surface and provide respective ultrasound images;
a remote system coupled to the sensing system through a communication link, the remote system comprising:
one or more non-transitory machine readable media to store data and instructions, the data comprising ultrasound image data representative of the respective ultrasound images provided by the ultrasound transducer modules, electrophysiological data representative of the electrophysiological signals measured from the body surface, and geometry data representing a spatial relationship between the electrophysiological sensors and patient anatomy in a three-dimensional coordinate system, the geometry data being determined based on the ultrasound image data; and
a processor to access the media and execute the instructions to perform a method comprising:
analyzing at least one of the ultrasound image data and the electrophysiological data; and
providing output data to visualize physiological information for the patient based on the analysis.

19. The system of claim 18, wherein the sensing system further comprises:
a sheet flexible material having a contact surface adapted to be placed on the outer surface of the patient's body;
a plurality of sensing apparatuses, each comprising:
a multimodal sensing apparatus comprising:
an instance of the ultrasound transducer module configured to at least sense acoustic energy from a transducer location of the sheet;
circuitry coupled to the transducer;
an instance of the electrophysiological sensor coupled to the circuitry, the sensor configured to at least sense electrophysiological signals from a sensor location of the sheet, in which the sensor location has a known spatial position relative to the transducer location; and
a monolithic substrate carrying the transducer, the circuitry and the electrophysiological sensor.

20. The system of claim 18, wherein the instructions executable by the processor are further programmed to calculate a metric that characterizes physiological information for the patient based on one or more of the electrophysiological data, the compounded three-dimensional image volume and reconstructed electrophysiological signals.

21. The system of claim 18, further comprising an interventional system comprising a device configured to perform an intervention at a site within the patient's body, wherein the instructions executable by the processor are further programmed to provide guidance associated with the intervention being provided based on one or more of the electrophysiological data and the compounded three-dimensional image volume.

22. The system of claim 18, wherein the instructions executable by the processor are further programmed to
reconstruct electrophysiological signals on an anatomical surface based on the electrophysiological data and the geometry data; and
provide output data to visualize the reconstructed electrophysiological signals on the anatomical surface.

23. The system of claim 22, wherein the anatomical surface comprises a three-dimensional or four-dimensional graphical representation generated based on the ultrasound image data.

24. A multimodal sensing apparatus, comprising:
a solid state transducer configured to at least sense acoustic energy from a transducer location;
circuitry coupled to the transducer;
an electrophysiological sensor coupled to the circuitry, the sensor configured to at least sense electrophysiological signals from a sensor location, in which the sensor location has a known spatial position relative to the transducer location; and
a monolithic substrate carrying the solid state transducer, the circuitry and the electrophysiological sensor.
